(19) United States
(12) Patent Application Publication
(10) Pub. No.: US 2010/0190184 A1
(43) Pub. Date: Jul. 29, 2010

(54) SCREENING METHOD UTILIZING NOVEL SUBSTRATE C-RET FOR GAMMA-SECRETASE

(75) Inventor: Eiji Inoue, Kobe-shi (JP)

Correspondence Address: DARBY & DARBY P.C., P.O. BOX 770, Church Street Station, New York, NY, 10008-0770, US

(73) Assignee: Eisai R & D Management Co., Ltd., Tokyo (JP)

(21) Appl. No.: 12/670,987

(22) PCT Filed: Aug. 1, 2008
(86) PCT No.: PCT/JP2008/063901

§ 371 Date: Jan. 27, 2010

Related U.S. Application Data
(60) Provisional application No. 60/953,230, filed on Aug. 1, 2007.

(51) Int. Cl. G01N 33/53

(52) U.S. Cl. 435/7.1

(57) ABSTRACT

The present invention provides a method of screening for compounds which affect the processing of c-Ret by γ-secretase. The method involves contacting a first biological composition containing γ-secretase or a biologically active fragment thereof with a second biological composition containing c-Ret in the presence and absence of a candidate compound; measuring the cleavage of the c-Ret in the presence and absence of the candidate compound; selecting those candidate compounds which affect the cleavage of the c-Ret by γ-secretase; and identifying the candidate compounds selected in the previous step as compounds which affect the processing of c-Ret by γ-secretase.

This is a U.S. National Phase Application under 35 U.S.C. §371 of International Patent Application No. PCT/JP2008/063901 filed Aug. 1, 2008, which claims the benefit of U.S. Provisional Patent Application No. 60/953,230 filed Aug. 1, 2007, both of which are incorporated by reference herein. The International Application was published in Japanese on Feb. 5, 2009 as WO2009/017234 A1 under PCT Article 21(2).

### TECHNICAL FIELD OF THE INVENTION

The present invention relates to a screening method using c-Ret, which is a novel substrate for γ-secretase, and a kit for use in the method.

### BACKGROUND OF THE INVENTION

γ-Secretase is a complex protein (aspartate protease) comprising presenilin, nicastrin, Aph-1 and Pen-2 as basic components. Presenilin is the catalytic domain. The presenilin gene has been identified as a causative gene for familial Alzheimer's disease (AD). γ-Secretase acts on single-pass transmembrane proteins as its substrates. As most representative substrates thereof, amyloid precursor protein (APP) and Notch are known. As a substance which specifically inhibits γ-secretase (γ-secretase inhibitor), (2S)-2-{[(3,5-Difluorophenyl)acetyl]amino}-N-[(3S)-1-methyl-2-oxo-5-phenyl-2,3-dihydro-1H-1,4-benzodiazepin-3-yl]propanamide is known (Hansson et al., J. Biol. Chem. (Dec. 3, 2004) Vol. 279 (Issue 49), pages 51654-51660; Zou et al., J. Biol. Chem. (Aug. 13, 2004) Vol. 279 (Issue 33), pages 34302-34310, Aug. 13, 2004). It is also known that APP produces amyloid β protein (Aβ) when cleaved by β-secretase at β-site and by γ-secretase at γ-site (Amtul et al., Neurobiology of Disease (Mar. 9, 2002) Vol. 2, pages 269-273). The thus-produced Aβ is classified into peptides with different lengths depending on the cleavage site in the amino acid sequence (C-terminal side). Of these peptides, Aβ42, which is strongly hydrophobic and ready to aggregate (ready to take the β-sheet structure), exhibits neurotoxicity. It has been considered that this phenomenon may be the major cause of Alzheimer's disease. Recently, however, a report has been made that presenilin 1 (PS1) and presenilin 2 (PS2) double-knockout mice capable of producing no Aβ show AD-like phenotypes such as decrease of synapses and neuronal death. This suggests existence of a pathogenic mechanism of AD independent from APP (Saura et al. Neuron. (Apr. 8, 2004) Vol. 42 (Issue 1), pages 23-36).

On the other hand, c-Ret is a member of the receptor tyrosine kinase family and is also known as one of GDNF (Glial-cell-line-derived neurotrophic factor) receptor subunits. It is known that GDNF signaling through c-Ret regulates cell differentiation and growth of cells; in particular, it has been revealed that this signaling shows protective effect on neurons in the central nervous system (Sariola et al. J Cell Sci. (Oct. 1, 2003) Vol. 116, pages 3855-62). However, it has never been reported to date that c-Ret is a substrate for γ-secretase.

### SUMMARY OF THE INVENTION

It is an object of the present invention to provide a screening method using c-Ret, a novel substrate for γ-secretase, in particular a method of screening for compounds which affect the processing of c-Ret by γ-secretase.

The present inventors have proved for the first time that c-Ret is cleaved by γ-secretase in HEK293 cells by using a γ-secretase inhibitor (2S)-2-{[(3,5-difluorophenyl)acetyl]amino}-N-[(3S)-1-methyl-2-oxo-5-phenyl-2,3-dihydro-1H-1,4-benzodiazepin-3-yl]propanamide. The present inventors have also found for the first time that it is possible to detect the cleavage of c-Ret by using an antibody specific to a hemagglutinin tag added at the C-terminus of c-Ret.

Briefly, the present inventors have demonstrated that a screening method with c-Ret utilizing the c-Ret degradation activity of γ-secretase (in particular, cleavage accelerating activity or cleavage inhibiting activity) is effective by showing that the cleavage of c-Ret is inhibited by γ-secretase inhibitor.

An accelerator for the c-Ret degradation activity of γ-secretase obtainable by the screening method of the present invention is a compound which accelerates the processing of c-Ret through γ-secretase. An inhibitor for the c-Ret degradation activity of γ-secretase obtainable by the screening method of the present invention is a compound which reduces the processing of c-Ret through γ-secretase. According to the present invention, it has become possible to develop therapeutics for memory disorders of interest (preferably AD) by selecting those compounds which act on γ-secretase selectively.

The present invention provides the following embodiments.

In one embodiment, the present invention relates to a method of screening for compounds which affect the processing of c-Ret by γ-secretase. More specifically, the method comprises the following steps: (a) a step of detecting the cleavage of c-Ret by γ-secretase, wherein a first biological composition containing γ-secretase or a biologically active fragment thereof is contacted with a second biological composition containing c-Ret to thereby measure the cleavage of the c-Ret; and (b) a step of evaluating whether or not candidate compounds affect the cleavage of c-Ret by γ-secretase, wherein those candidate compounds which affect the cleavage of the c-Ret by γ-secretase are selected and the thus selected compounds are identified as compounds which affect the processing of c-Ret by γ-secretase.

In another embodiment, the present invention relates to a screening method further comprising, in addition to the above-described steps, a step of evaluating a candidate compound as a compound which inhibits the processing of c-Ret through γ-secretase or an inhibitor for the c-Ret degradation activity of γ-secretase, when c-Ret undegraded product in the presence of the candidate compound was increased relative to c-Ret undegraded product in the absence of the candidate compound.

In still another embodiment, the present invention relates to a screening method further comprising, in addition to the above-described steps, a step of evaluating a candidate compound as a compound which accelerates the processing of c-Ret through γ-secretase or an accelerator for the c-Ret degradation activity of γ-secretase, when c-Ret undegraded product in the presence of the candidate compound was decreased relative to c-Ret undegraded product in the absence of the candidate compound.

In still another embodiment, the present invention relates to a screening method further comprising, in addition to the above-described steps, a step of evaluating a candidate compound as a compound which accelerates the processing of c-Ret through γ-secretase or an accelerator for the c-Ret degradation activity of γ-secretase, when c-Ret cleavage product in the presence of the candidate compound was increased relative to c-Ret cleavage product in the absence of the candidate compound.

In still another embodiment, the present invention relates to a screening method further comprising, in addition to the above-described steps, a step of evaluating a candidate compound as a compound which inhibits the processing of c-Ret through γ-secretase or an inhibitor for the c-Ret degradation activity of γ-secretase, when c-Ret cleavage product in the presence of the candidate compound was decreased relative to c-Ret cleavage product in the absence of the candidate compound.

In still another embodiment, the present invention relates to a screening method further comprising, in addition to the above-described method, a step of measuring the cleavage of APP or a polypeptide comprising a γ-secretase cleavage site of APP (hereinafter, expressed as “polypeptide comprising an APP γ-secretase cleavage site”).

In still another embodiment, the present invention relates to a screening method further comprising, in addition to the above-described method, a step of measuring the cleavage of Notch or a polypeptide comprising a γ-secretase cleavage site of Notch (hereinafter, expressed as “polypeptide comprising a Notch γ-secretase cleavage site”).

In still another embodiment, the present invention provides a pharmaceutical composition comprising at least one compound identified by the screening method of the present invention and a pharmacologically acceptable carrier. Preferably, the above compound is (2S)-2-{[(3,5-difluorophenyl)acetyl]amino}-N-[(3S)-1-methyl-2-oxo-5-phenyl-2,3-dihydro-1H-1,4-benzodiazepin-3-yl]propanamide (hereinafter, sometimes referred to as “Compound E”).

In still another embodiment, the present invention provides a method of treating a disease or condition characterized by abnormality in the processing of c-Ret by γ-secretase, comprising a step of administering to a patient in need of treatment (e.g., a patient with a memory disorder, especially a condition of dementia (preferably AD)) an effective dose of the compound of the present invention or a pharmaceutical composition comprising the same (preferably a therapeutic for dementia), wherein preferably the dose is effective for altering the c-Ret processing activity of γ-secretase in the cells of the patient.

In still another embodiment, the present invention provides a test kit for measuring the processing of c-Ret by γ-secretase, which is applicable to the method of the present invention, comprising c-Ret and preferably further comprising a substrate for γ-secretase other than c-Ret (preferably APP and/or Notch).

In still another embodiment, the present invention provides a test kit for measuring the processing of c-Ret by γ-secretase, comprising a first biological composition containing γ-secretase or a biologically active fragment thereof and a second biological composition containing c-Ret.

According to the present invention, there is provided a method of screening for compounds which affect the processing of c-Ret by γ-secretase. The compound screened by the present invention is useful as a therapeutic for a memory disorder of interest, especially dementia (preferably AD).

### BRIEF DESCRIPTION OF THE DRAWINGS

FIG. 1 is a diagram showing the results of analysis of c-Ret processing using c-Ret-transfected 293/EBNA-1 cell strain.

### DETAILED DESCRIPTION OF THE INVENTION

Hereinbelow, the present invention will be described in more detail. The present specification encompasses the contents disclosed in the specification and the drawings of U.S. Provisional Patent Application No. 60/953,230 (filed on Aug. 1, 2007) based on which the present patent application claims priority.

The term “patient” used herein refers to an animal, preferably a mammal The term “mammal” used herein means any animal classified as mammal, including human and non-human mammals (such as mouse, rat, hamster, guinea pig, rabbit, pig, dog, horse, cattle, monkey, etc.). Preferably, the mammal in the present specification is human. When the mammal is human, the term “patient” include adults and children, and also male and female. Children include newborn infants, infants and adolescents.

The term “γ-secretase” used herein means an enzyme or a complex composed of a plurality of molecules, each of which is in charge of the production of Aβ by cleaving (degrading) APP within its transmembrane domain. The plurality of molecules comprise at least one molecule selected from presenilin, nicastrin, Aph-1 and Pen-2. Examples of the γ-secretase of the present invention include mouse Presenilin 1(NM_{—}008943), rat Presenilin1 (D82363), human Presenilin1 (NM_{—}000021), mouse Presenilin2 (NM_{—}011183), rat Presenilin2 (NM_{—}031087), human Presenilin2 (NM_{—}000447), mouse Nicastrin (NM_{—}021607), rat Nicastrin (NM_{—}174864), human Nicastrin (NM_{—}015331), mouse Aph-1 (NM_{—}146104), rat Aph-1 (NM_{—}001014255), human Aph-1 (NM_{—}016022), mouse Pen-2 (NM_{—}025498), rat Pen-2 (NM_{—}001008764) and human Pen-2 (NM_{—}172341). Each component molecule of the γ-secretase of the present invention may be a full-length molecule or a part thereof, as long as that γ-secretase has an enzyme activity equivalent to that of γ-secretase functioning in vivo. Further, the γ-secretase of the present invention may be a mutant γ-secretase. The mutant γ-secretase is a polypeptide composed of full-length component molecules which may have deletion, substitution, insertion and/or addition of one or more (preferable one or several) amino acids, or a polypeptide having an amino acid sequence comprising a combination of such mutations, each of the above polypeptide having an enzyme activity equivalent to that of γ-secretase functioning in vivo.

The term “biologically active fragment of γ-secretase” means a fragment having an enzyme activity equivalent to that of γ-secretase functioning in vivo. Examples of such fragments include fragments capable of cleaving APP or c-Ret.

It should be noted that sometimes, the term “γ-secretase” is intended to include the “biologically active fragment of γ-secretase” in the present specification. The term “c-Ret” used herein refers to a polypeptide which is a member of the receptor tyrosine kinase family and is also known as one of GDNF (Glial-cell-line-derived neurotrophic factor) receptor subunits (Sariola H, and Saarma M., Novel functions and signalling pathways for GDNF, J Cell Sci. 2003 Oct. 1; 116:3855-62).

The c-Ret used in the present invention is preferably a c-Ret derived from a mammal. For example, human c-Ret (NM_{—}020975, X12949, M57464, NM_{—}020630, BC004257), rat c-Ret (NM_{—}012643, AJ299017), mouse c-Ret (NM_{—}009050, X67812, NM_{—}001080780, AK044686), dog (Canis lupus familiaris) c-Ret (XM_{—}543915), chicken (Gallus gallus) c-Ret (NM_{—}205190), platypus (Ornithorhynchus anatinus) c-Ret (XM_{—}001507665), African clawed frog (Xenopus laevis) c-Ret (NM_{—}001085623) and the like are included in the c-Ret of the present invention.

Genes encoding the above-listed c-Rets and the corresponding c-Ret polypeptides are included in the c-Ret of the present invention. Table 1 below shows correspondence between various animal-derived c-Ret polypeptides and their nucleotide and amino acid sequences.

TABLE 1

| | | Nucleotide | |
|---|---|---|---|
| Source or Type | Accession No. | Sequence | Amino Acid Sequence |
| Human | NM_020975 | SEQ ID NO: 1 | SEQ ID NO: 2 |
| (Homo sapiens) | X12949 or M57464 | SEQ ID NO: 3 | SEQ ID NO: 4 |
| | NM_020630 | SEQ ID NO: 5 | SEQ ID NO: 6 |
| | BC004257 | SEQ ID NO: 7 | SEQ ID NO: 8 |
| Rat | NM_012643 | SEQ ID NO: 9 | SEQ ID NO: 10 |
| (Rattus norvegicus) | AJ299017 | SEQ ID NO: 11 | SEQ ID NO: 12 |
| Rat-HA | | SEQ ID NO: 13 | SEQ ID NO: 14 |
| (Rattus norvegicus) | | | |
| Mouse | NM_009050 | SEQ ID NO: 15 | SEQ ID NO: 16 |
| (Mus musculus) | X67812 | SEQ ID NO: 17 | SEQ ID NO: 18 |
| | NM_001080780 | SEQ ID NO: 19 | SEQ ID NO: 20 |
| | AK044686 | SEQ ID NO: 21 | SEQ ID NO: 22 |
| Dog | XM_543915 | SEQ ID NO: 23 | SEQ ID NO: 24 |
| (Canis lupus familiaris) | | | |
| Chicken | NM_205190 | SEQ ID NO: 25 | SEQ ID NO: 26 |
| (Gallus gallus) | | | |
| Platypus | XM_001507665 | SEQ ID NO: 27 | SEQ ID NO: 28 |
| (Ornithorhynchus | | | |
| anatinus) | | | |
| African clawed frog | NM_001085623 | SEQ ID NO: 29 | SEQ ID NO: 30 |
| (Xenopus laevis) | | | |

In the present invention, c-Ret structurally comprises a cadherin domain, a transmembrane domain and a kinase activity site (Sariola H et al., J Cell Sci. 2003 Oct. 1; 116:3855-62). The ligand of c-Ret is GDNF (Sariola H et al., J Cell Sci. 2003 Oct. 1; 116:3855-62). The c-Ret of the present invention may be either a full-length polypeptide or a partial sequence thereof, as long as it comprises the γ-secretase cleavage site of c-Ret.

Further, in the present invention, c-Ret may be a mutant c-Ret. The mutant c-Ret is a full-length c-Ret polypeptide which may have deletion, substitution, insertion and/or addition of one or more (preferable one or several) amino acids, or a c-Ret polypeptide having an amino acid sequence comprising a combination of such mutations, each of the above polypeptides being functionally substantially identical with c-Ret.

The “polypeptide which is functionally substantially identical with c-Ret” means a polypeptide having an activity of c-Ret, for example, a polypeptide which has a cleavage activity in a γ-secretase-dependent manner, and includes any and all c-Ret derivatives comprising at least the γ-secretase cleavage site of c-Ret. These polypeptides are especially useful in detecting and purifying c-Ret.

The term “substitution” used herein means preferably conservative substitution in which one or more (preferably one or several) amino acid residues are substituted with other chemically similar amino acid residues so that the activity of the peptide is not substantially modified. Examples of conservative substitution include substitution of a hydrophobic residue with other hydrophobic residue and substitution of a polar residue with other polar residue with the same electric charge. Functionally similar amino acids which allow such substitution are known to those skilled in the art for each amino acid. Specifically, examples of non-polar (hydrophobic) amino acids include alanine, valine, isoleucine, leucine, proline, tryptophan, phenylalanine and methionine; examples of polar (neutral) amino acids include glycine, serine, threonine, tyrosine, glutamine, asparagine and cystein. Examples of positively charged (basic) amino acids include arginine, histidine and lysine. Examples of negatively charged (acidic) amino acids include aspartic acid and glutamic acid.

The number of amino acids which may be deleted, substituted, inserted and/or added as described above is, for example, 1 to 30, preferably 1 to 20, more preferably 1 to 10, still more preferably 1 to 5, particularly preferably 1 to 2.

The above-described identity means that the amino acid sequence of a polypeptide may not be identical with the amino acid sequence of c-Ret as long as the polypeptide has substantially the same function as that of c-Ret. Therefore, when a polypeptide has an activity functionally substantially the same as that of c-Ret (e.g., γ-secretase-dependent degradation activity), the degree of homology of the amino acid sequence of the relevant polypeptide is disregarded. The animal species from which the polypeptide is derived is also disregarded.

In a preferred embodiment of the present invention, a polypeptide functionally substantially identical to c-Ret is an amino acid sequence which has preferably 80% or more, more preferably 85% or more, still more preferably 90% or more, still more preferably 95% or more, particularly preferably 98% or more, most preferably 99% or more homology to the amino acid sequence of c-Ret (e.g., the amino acid sequence of a polypeptide consisting of SEQ ID NO: 10 (rat c-Ret)) and has substantially the same activity as that of c-Ret. When a polypeptide has these features, the polypeptide may be any of a polypeptide derived from the above-listed animals, a recombinant polypeptide or a synthetic polypeptide.

The identity described above may be values calculated by homology search programs known to those skilled in the art. For example, identity can be calculated by using default parameters in the homology algorithm BLAST (Basic local alignment search tool; http://www.ncbi.nlm nih.gov/BLAST/) of The National Center for Biotechnology Information (NCBI).

The c-Ret may take any of the following forms: a fusion polypeptide fused to other polypeptide, a tagged or labeled polypeptide or a polypeptide otherwise modified. These polypeptides may be obtained by recombinant DNA techniques, site-directed mutagenesis, treatment with mutagenic agents (such as hydroxylamine), or automated peptide synthesis.

Examples of particularly useful systems as tagged c-Ret polypeptides include hemagglutinin (HA) system, glutathione-S-transferase (GST) system, maltose-binding protein system, 6× histidine system, 8× histidine system and the like.

Examples of modification incorporating the above-mentioned label or other detectable moieties include biotin label, radioactive label, fluorescence label, chemiluminescence label and the like. The c-Ret of the present invention may be labeled with one, two or more of these labels.

In a preferred embodiment of the present invention, c-Ret is a rat c-Ret, for example, a polypeptide comprising the amino acid sequence as shown in SEQ ID NO: 10 or SEQ ID NO: 12. In a still preferred embodiment of the present invention, c-Ret is a rat c-Ret to which an HA tag is added. For example, a rat c-Ret polypeptide with an HA tag added at its C-terminus (SEQ ID NO: 14) may be used (Example 1). Polypeptides comprising the entire human c-Ret amino acid sequence or a part thereof (e.g., polypeptides comprising the amino acid sequence as shown in SEQ ID NO: 2, 4, 6 or 8) or c-Ret polypeptide sequences derived from other animals listed in Table 1 may also be used in the same manner as rat c-Ret.

The present invention further provides a polynucleotide comprising a nucleotide sequence encoding the above-described c-Ret. The polynucleotide encoding the c-Ret of the present invention is a polynucleotide encoding a polypeptide having substantially the same activity as that of c-Ret (e.g., γ-secretase-dependent degradation activity). In a preferred embodiment of the present invention, the polynucleotide encoding the c-Ret of the present invention is a polynucleotide encoding a rat c-Ret, e.g., the polynucleotide as shown in SEQ ID NO: 9 or SEQ ID NO: 11. Further, in a preferred embodiment of the present invention, the polynucleotide encoding the c-Ret of the present invention is a polynucleotide encoding a rat c-Ret polypeptide with an HA tag added. For example, a polynucleotide encoding a rat c-Ret polypeptide to which an HA tag is added at its C-terminus (SEQ ID NO: 13) may be given (Example 1). Polynucleotides comprising the entire human c-Ret nucleotide sequence or a part thereof (e.g., the nucleotide sequence as shown in SEQ ID NO: 1, 3, 5 or 7) or nucleotide sequences derived from other animals listed in Table 1 may also be used in the same manner as rat c-Ret polynucleotides.

The polynucleotide encoding the c-Ret of the present invention may be a polynucleotide encoding the above-described c-Ret mutant or c-Ret derivative. For example, a polynucleotide which comprises a nucleotide sequence having 80% or more, preferably 85% or more, still more preferably 90% or more, yet still more preferably 95% or more, particularly preferably 98% or more, most preferably 99% or more homology (identity) with the nucleotide sequence as shown in SEQ ID NO: 9 or SEQ ID NO: 11 and encodes a polypeptide having substantially the same activity as that of c-Ret may be included.

Further, the polynucleotide encoding the c-Ret of the present invention includes a polynucleotide which hybridizes to a polynucleotide consisting of a nucleotide sequence complementary to the nucleotide sequence as shown in SEQ ID NO: 9 or SEQ ID NO: 11 under stringent conditions and yet encodes a polypeptide having substantially the same activity as that of c-Ret. The stringent conditions refer to, for example, “2×SSC, 0.1% SDS, 42° C.” or “1×SSC, 0.1% SDS, 37° C.” as washing conditions after hybridization. As more stringent conditions, “1×SSC, 0.1% SDS, 65° C.” or “0.5×SSC, 0.1% SDS, 50° C.” may be given, for example. More specifically, a method using Rapid-hyb buffer (Amersham Life Science) may be employed in which pre-hybridization is performed at 68° C. for more than 30 minutes; then, with addition of a probe, a hybrid is formed while keeping the reaction solution at 68° C. for more than 1 hour, followed by washing in 2×SSC, 0.1% SDS at room temperature for 20 minutes 3 times, washing in 1×SSC, 0.1% SDS at 37° C. for 20 minutes 3 times and finally washing in 1×SSC, 0.1% SDS at 50° C. for 20 minutes twice. Alternatively, other method may be employed in which pre-hybridization is performed in Expresshyb Hybridization Solution (CLONTECH) at 55° C. for more than 30 minutes; then, with addition of a labeled probe, the reaction solution is incubated at 37-55° C. for more than 1 hour, followed by washing in 2×SSC, 0.1% SDS at room temperature for 20 minutes 3 times, washing in 1×SSC, 0.1% SDS at 37° C. for 20 minutes once. In these methods, more stringent conditions may be achieved, for example, by raising the temperature of prehybridization, hybridization or the second washing. For example, the temperatures of prehybridization and hybridization may be raised to 60° C., respectively; for more stringent conditions, the temperatures may be raised to 68° C., respectively. Those skilled in the art could appropriately select conditions for obtaining c-Ret isoforms, allelic mutants, and corresponding genes derived from other organisms, by taking into account various conditions such as probe concentration, probe length, reaction time, etc. in addition to the above-described salt concentrations and reaction temperatures.

Such polynucleotides may be obtained by gene amplification techniques, hybridization techniques, recombinant DNA techniques, and the like.

The term “biological composition” used herein means a composition comprising γ-secretase or a biologically active fragment thereof, or c-Ret, and is not particularly limited. For example, the biological composition may be a cell-free reconstruction system, a mammal or a part thereof, or a transgenic non-human mammal so engineered to overexpress APP or a part of this transgenic mammal.

In the expressions “first biological composition containing γ-secretase or a biologically active fragment thereof” and “second biological composition containing c-Ret” used herein, the γ-secretase and/or c-Ret may be either endogenous or exogenous.

When the γ-secretase or c-Ret is endogenous, the composition may be any composition as long as it contains γ-secretase or c-Ret derived from the above-mentioned animal or a part thereof.

The term “a part of the above-mentioned animal” includes tissues, cells, cell lysates, cell membrane fractions or purified membranes of the above-mentioned animal. As examples of the cells, cells in the central nervous system; neurons such as brain-derived neurons, cerebral cortex-derived neurons, cerebral cortex-derived primarily cultured neurons, or hippocampus-derived primarily cultured neurons; or glial cells may be enumerated. The γ-secretase or c-Ret may be in the state of being contained in a mammal or a part thereof. Alternatively, the γ-secretase or c-Ret may be a γ-secretase fraction or a c-Ret fraction of cell lysate prepared from a mammal. The cell lysate may be obtained by subjecting γ-secretase- or c-Ret-containing cells to lysis with a hypotonic solution or surfactant, or to sonication or other physical disruption. Optionally, the cell lysate may be purified with some purification means such as columns.

When the γ-secretase or c-Ret is exogenous, the biological composition may be γ-secretase expressing cells or c-Ret expressing cells prepared by allowing host cells to express the whole or a part of the sequences in expression vectors comprising a polynucleotide encoding the individual molecules constituting γ-secretase or a polynucleotide encoding c-Ret. Alternatively, the biological composition may be the γ-secretase fraction of a cell lysate derived from γ-secretase expressing cells, or the c-Ret fraction or cell membrane fraction of a cell lysate derived from c-Ret expressing cells. The cell lysate may be obtained by subjecting γ-secretase- or c-Ret-containing cells to lysis with a hypotonic solution or surfactant, or to sonication or physical disruption. Optionally, the cell lysate may be purified with some purification means such as columns. The expression vector may be a vector which is transformed or transfected into a host cell and temporarily expresses the gene of interest. Alternatively, the expression vector may be a vector which is integrated into the genome of a host cell and expresses the gene of interest stably.

The term “transformation” or “transfection” used herein means any and all methods which change DNA contents in eukaryotic cells or microorganisms. These methods include calcium phosphate transfection, protoplast fusion transfection, electroporation transfection, DEAE-dextran transfection, liposome transfection, polybrene transfection and direct microinjection transfection (Sambrook, et al., Molecular Cloning 3: 16.30-16.31 (1989)).

The host cell into which the above-described expression vector is to be transformed or transfected may be any cell (or cell line) or microorganism capable of expressing the gene of interest. Known cultured cells or microorganisms may be used as host cells. Examples of mammal cells or cell lines which may be used as host cells include HEK 293 cells, Chinese hamster ovary (CHO) cells, fibroblast cells, primary endothelial cells (HUVEC cells), human glioma cells, HeLa cells, COS cells, PC12 cells, lymphoblast cells, melanoma cells, hybridoma cells, oocytes and embryonic stem cells. Examples of known microorganisms which may be used as host cells include Escherichia coli and yeast. Further, insect cells such as BmN4 cells may also be used.

The expression vector used in the above-described transformation or transfection is not particularly limited, as long as the vector comprises a polynucleotide encoding the individual molecules constituting γ-secretase or a polynucleotide encoding c-Ret. Such an expression vector may be a plasmid obtainable by introducing the polynucleotide into a known expression vector selected appropriately depending on the host cell to be used.

Examples of the above-mentioned expression vector include pUC, pTV, pGEX, pKK or pTrcHis for E. coli; pEMBLY or pYES2 for yeast; pcDNA3, pMAMneo and pBabe Puro for CHO cells, HEK293 cells or COS cells; and a vector comprising the polyhedrin promoter of Bombyx mori nuclear polyhedrosis virus (BmNPV) (such as pBK283) for BmN4 cells.

In mammal cells, examples of promoters which give a strong transcription activity include CMV immediate early promoter, retrovirus promoters (e.g., LTR from MLU or MMTV), promoters of SV40, RSV LTR, HIV-1 LTR and HIV-2 LTR, adenovirus promoters (e.g., those from E1A region, E2A region or MLP region), and promoters of AAV LTR, cauliflower mosaic virus, HSV-TK and avian sarcoma virus.

The above-described transformed or transfected host cell is not particularly limited, as long as the host cell comprises a polynucleotide encoding the individual molecules constituting γ-secretase or a polynucleotide encoding c-Ret. For example, the transformed cell may be a transformant in which the polynucleotide has been integrated into the chromosome thereof. Alternatively, the transformed cell may be a transformant comprising the polynucleotide in the form of a plasmid. It is also possible that the transformed cell is a transformant which is not expressing γ-secretase or c-Ret. These transformants may be obtained by transforming a desired host cell with the above-mentioned plasmid or the above-described polynucleotide per se.

Cells containing the above-described γ-secretase and/or c-Ret are not particularly limited as long as the cell is capable of expressing γ-secretase and/or c-Ret on the surface of its cell membrane. As examples of such cells, a cell expressing endogenous γ-secretase and endogenous c-Ret, a cell expressing γ-secretase and c-Ret one of which is endogenous and the other is exogenous or a cell expressing exogenous γ-secretase and exogenous c-Ret may be given. Such cells may also be obtained by culturing under conditions which allow expression of γ-secretase and/or c-Ret. Alternatively, such cells may be obtained by injecting into an appropriate cell an RNA encoding the individual molecules constituting γ-secretase and/or an RNA encoding c-Ret and culturing the resultant cell under conditions which allow expression of γ-secretase and/or c-Ret.

The above-described cell membrane fraction may be obtained, for example, by disrupting cells expressing the γ-secretase or c-Ret of the present invention and isolating cell membrane-rich fractions. As methods for disrupting cells, homogenizing in a homogenizer; disrupting in a Waring blender or Polytron; disrupting by sonication; ejecting cells from a thin nozzle while applying pressure with a French press; and so on may be used. As methods for fractionating cell membrane, fractionation methods with centrifugal force such as differential centrifugation or density gradient centrifugation may be used.

For purification, a known method for protein purification may be used. The method comprises a step of crudely fractionating cells into polypeptide fractions and non-polypeptide fractions. After the γ-secretase or c-Ret of the present invention has been isolated from other polypeptides with a column or the like, the desired γ-secretase or c-Ret is further purified by chromatography or electrophoresis to thereby achieve partial purification or complete purification (or homogeneity by purification). Examples of analysis methods particularly suitable for preparation/purification of pure peptides include precipitation using ammonium sulfate, PEG, antibodies, etc.; centrifugation after thermal denaturation; chromatography step; isoelectric focusing; gel electrophoresis; SDS (sodium dodecyl sulfate)-polyacrylamide electrophoresis (SDS-PAGE); and a combination of these methods and other method. Examples of chromatography step includes ion exchange chromatography, gel filtration chromatography, reversed-phase chromatography, hydroxyapatite chromatography, affinity chromatography, fast protein liquid chromatography (FPLC), high performance liquid chromatography (HPLC) and immobilized metal ion affinity chromatography (IMAC).

Alternatively, γ-secretase or c-Ret may be tagged in advance; then, a crude polypeptide may be applied to a purification column to which a protein that recognizes the tag has been bound; the desired γ-secretase or c-Ret adsorbed onto the column may be desorbed from the column by feeding an appropriate solvent thereinto.

Various purification steps may be performed in a different order, or some of the steps may be omitted. A preferable method for evaluating the purity of a fraction is a method in which the specific activity of the fraction is calculated and compared with the specific activity of the first extract, followed by calculation of the magnitude of purity for evaluation.

The term “cleavage of c-Ret” used herein refers to an event in which c-Ret, cut by γ-secretase, produces a fragment shorter than the initial c-Ret before cutting. The term “c-Ret undegraded product” used herein refers to a polypeptide which is produced as a result of non-cleavage of c-Ret; this polypeptide means the c-Ret polypeptide before degradation by γ-secretase.

For monitoring the cleavage of c-Ret by γ-secretase, any of the following antibodies may be used: an anti-c-Ret antibody; an antibody which recognizes c-Ret undegraded product produced as a result of non-cleavage of c-Ret; or an antibody which recognizes c-Ret cleaved product produced as a result of cleavage of c-Ret, preferably an antibody which recognizes the intracellular domain of c-Ret, more preferably an antibody which recognizes the C-terminal domain of c-Ret. When the undegraded product or cleaved product of a tagged c-Ret polypeptide is to be detected, an antibody which recognizes the selected tag may be used. For example, when an HA tag has been added to the C-terminus of c-Ret, it is possible to detect the undegradation or cleavage of c-Ret using an anti-HA tag antibody. In this case, the antibody is capable of clarifying the presence and concentration of a C-terminal fragment of c-Ret that is produced as a result of non-cleavage of c-Ret or the presence and concentration of a C-terminal fragment of c-Ret that is produced as a result of cleavage of c-Ret.

The term “APP” used herein β-amyloid precursor protein (βAPP) or a mutant thereof. APP is a single-pass transmembrane protein comprising an Aβ domain in its C-terminal region, expressed in a large variety of cells in many mammals. In human, APP is encoded by the gene APP located in the long arm of chromosome No. 21 and has three major isotypes (APP695, APP751 and APP770). APP695, APP751 and APP770 consist of 695, 751 and 770 amino acid residues, respectively. Examples of APP proteins include human APP695 (NM_{—}201414, NP_{—}958817 and P05067-4), human APP751 (NM_{—}201413, NP_{—}958816 and P05067-8), human APP770 (NM_{—}000484, NP_{—}000475, P05067-1 and P05067), mouse APP695 (NM_{—}007471, NP_{—}031497 and P12023-2), mouse APP751 (P12023-3), mouse APP770 (AY267348, AAP23169, P12023-1 and P12023), rat APP695 (P08592-2), rat APP751 (P08592-7) and rat APP770 (NM_{—}019288, NP_{—}062161, P08592-1 and P08592). Examples of APP mutants include Swedish FAD double mutant, London mutant, valine717 to phenylalanine mutant, valine717 to isoleucine mutant and valine717 to glycine mutant.

The term “Aβ” used herein means the term β-amyloid protein, amyloid β protein, β-amyloid peptide, amyloid β peptide or amyloid beta. For example, Aβ is a peptide consisting of about 33-44 amino acids residues in human APP695 amino acid isotype. Preferably, Aβ includes any peptide comprising a part or all of the amino acid residues from positions 597 to 640 in APP, and means every peptide produced from APP by its N-terminal protein degradation and subsequent C-terminal protein degradation. Aβ40 and Aβ42 are peptides comprising 40 amino acid residues and 42 amino acid residues, respectively.

The term “Notch” used herein refers to one of the competing substrates for γ-secretase belonging to the cell surface receptor Notch family. For example, human Notch 1 (AF308602.1), mouse Notch 1 (NM_{—}008714.2) and rat Notch 1 (N_{—}001105721.1) may be enumerated. Since Notch 1 has an important function in hematopoiesis, inhibition of the processing of Notch 1 may potentially cause immunodeficiency and anemia.

The term “candidate compound” used herein means a compound which is tested in the compound screening method. Although every molecule may be used as a candidate compound, preferably a compound capable of changing the activity of γ-secretase (preferably the activity of γ-secretase of mammals) is used. The candidate compound is one or more compounds contained in expression products of gene libraries; natural or synthetic low molecular weight compound libraries; nucleic acids (oligo DNA or oligo RNA); natural or synthetic peptide libraries; antibodies; substances released from bacteria (including those released from bacteria as a result of metabolism); cell (from microorganisms, plant cells or animal cells) extracts; cell (microorganism, plant cell or animal cell) culture supernatants; purified or partially purified peptides; extracts from marine organisms, plants or animals; soils; or random phage peptide display libraries. The above-described compound may be either a novel compound or a known compound. Further, the above-described compound may be a compound modified by conventional chemical means, physical means and/or biochemical means. For example, the above-described compound may be a structural analogue which is obtained by subjecting the initial compound to direct chemical modification (such as acylation, alkylation, esterification or amidation) or random chemical modification. The candidate compound may also be a compound identified by pharmacophore search of the above-described compound or structure comparison programs with computer. The candidate compound may be in the form of a salt. Further, the candidate compound or a salt thereof may be in the form of a solvate (including hydrate).

Further, the candidate compound may be a known γ-secretase accelerator or γ-secretase inhibitor involved in the processing of APP and/or the processing of Notch, or a structural analogue of the above accelerator or inhibitor. A known compound which accelerates or inhibits the activity of γ-secretase and/or the processing of APP and/or the processing of Notch may be a compound that can be designed through rational drug design. For example, DAPT (N—[N-(3,5-difluorophenacetyl)-L-alanyl]-S-phenylglycine t-butyl ester), CM256 (N-[(1,1dimethylethoxy)carbonyl]-L-valyl-(4S,5S)-4-amino-2,2-difluoro-5-methyl-3-oxoheptanoyl-L-valyl-L-lsoleucine methyl ester) and (2S)-2-{[(3,5-difluorophenyl)acetyl]amino}-N-[(3S)-1-methyl-2-oxo-5-phenyl-2,3-dihydro-1H-1,4-benzodiazepin-3-yl]propanamide (Alexis Biochemicals) may be enumerated.

The expression “compound which affects the processing of c-Ret by γ-secretase” used herein means either a compound which inhibits the c-Ret cleavage activity by γ-secretase (γ-secretase inhibitor) or a compound which accelerates the c-Ret cleavage activity by γ-secretase (γ-secretase accelerator). It should be noted that γ-secretase inhibitor includes antagonist to γ-secretase and that γ-secretase accelerator includes agonist to γ-secretase. γ-Secretase inhibitor and γ-secretase accelerator also include those compounds which alter the cleavage site of c-Ret cleavage products by γ-secretase to thereby produce c-Ret cleavage products with different peptide lengths.

The term “salt” used herein refers to a pharmacologically acceptable salt, and is not particularly limited as long as it forms a pharmacologically acceptable salt with the above-described compound. Preferred examples thereof are hydrohalogenic acid salts (such as hydrofluoride, hydrochloride, hydrobromide or hydroiodide), inorganic acid salts (such as sulfate, nitrate, perchlorate, phosphate, carbonate or hydrogencarbonate), organic carboxylic acid salts (such as acetate, oxalate, maleate, tartrate, fumarate or citrate), organic sulfonic acid salts (such as methanesulfonate, trifluoromethanesulfonate, ethanesulfonate, benzenesulfonate, toluenesulfonate or camphorsulfonate), amino acid salts (such as aspartate or glutamate), quaternary amine salts, alkali metal salts (such as lithium salt, sodium salt or potassium salt) and alkaline earth metal salts (such as magnesium salt or calcium salt).

According to the present invention, there are provided (a) an assay method for examining the cleavage of c-Ret and (b) a method of evaluating whether or not a candidate compound is a compound which affects γ-secretase (screening method) utilizing the assay method. Briefly, the following method is provided as the method of the present invention.

The method of screening for compounds which affect the processing of c-Ret by γ-secretase (which is an embodiment of the present invention) comprises the following steps:

(i) contacting a first biological composition containing γ-secretase or a biologically active fragment thereof with a second biological composition containing c-Ret in the presence and absence of a candidate compound;
(ii) measuring the cleavage of the c-Ret in the presence and absence of the candidate compound;
(iii) selecting those candidate compounds which affect the cleavage of the c-Ret by γ-secretase; and
(iv) identifying the candidate compounds selected in step (iii) as compounds which affect the processing of c-Ret by γ-secretase.

The method of the present invention may be performed in an appropriate cell system containing γ-secretase and c-Ret or a cell-free system containing γ-secretase and c-Ret.

The cell system containing γ-secretase and c-Ret may be either a cell system expressing endogenous genes or a cell system containing an exogenous gene(s). It is possible to contact a first biological composition containing γ-secretase with a second biological composition containing c-Ret by culturing a cell containing γ-secretase and c-Ret in an appropriate medium in the presence and absence of a candidate compound and incubating the cell under reaction conditions which allow the cleavage of c-Ret by γ-secretase activity. If a cell system containing an exogenous gene(s) is used, the above-described contact may be performed under culture conditions which allow the expression of the exogenous gene. It is also possible to apply conditions that allow the cleavage of other γ-secretase substrates, e.g., reaction conditions known to those skilled in the art when the substrate is APP.

Examples of reaction conditions are enumerated below. For cell systems expressing endogenous genes, in the case of primary culture of neurons, culture conditions are in MEM (Invitrogen) medium supplemented with 5% FBS (Hyclone), 1× B27 Supplement (Invitrogen), 0.5 mM L-glutamine (Invitrogen), 25 μg/ml insulin (SIGMA) and 8 μM AraC (SIGMA); under 5% CO₂; and at 37° C. For cell systems containing an exogenous gene(s), in the case of HEK293 cell line, culture conditions are in 10% FBS (Hyclone)/DMEM (Invitrogen), under 5% CO₂ and at 37° C.

In cell-free systems, a first biological composition containing γ-secretase or a biologically active fragment thereof (e.g., cell membrane fraction containing γ-secretase) and a second biological composition containing c-Ret (e.g., cell membrane fraction containing c-Ret) may be contacted with each other by incubating these compositions by mixing them in the presence and absence of a candidate compound. These compositions may be mixed under reaction conditions which allow the cleavage of c-Ret by γ-secretase activity, e.g., 10 mM HEPES, pH 7.4, 150 mM NaCl, 10% glycerol, 5 mM EDTA, 5 mM 1,10-phenanthroline, 10 μg/ml phosphoramidon, Complete protease inhibitor cocktail (Roche Biochemicals) (Tomita et al., Molecular Neurodegeneration 2006 1:2). Alternatively, these compositions may be mixed under conditions which allow the cleavage of other γ-secretase substrates, e.g., reaction conditions known to those skilled in the art when the substrate is APP. γ-Secretase or c-Ret may be a purified γ-secretase or c-Ret, a biologically active fragment of γ-secretase or c-Ret, an analogue of γ-secretase or c-Ret, or a mutant of γ-secretase or c-Ret.

The candidate compound may be added generally within a range from approx. 1 nM to 1 mM, usually within a range from approx. 10 μM to 1 mM.

By contacting a first biological composition containing γ-secretase with a second biological composition containing c-Ret as described above, c-Ret cleavage reaction by γ-secretase occurs.

In order to identify a compound which changes the cleavage of c-Ret by γ-secretase, the steps described above are performed in the presence and absence of a candidate compound. Then, the c-Ret cleavage activity of γ-secretase in the presence of the candidate compound is compared with that activity in the absence of the candidate compound (control) to evaluate the effect of the candidate compound upon cleavage activity. By these procedures, it is possible to identify compounds which change the cleavage of c-Ret by γ-secretase.

Even a slight change in the quantity or degree of c-Ret in the presence of a candidate compound indicates that the c-Ret cleavage activity of γ-secretase has been changed in the presence of the candidate compound. Therefore, the candidate compound can be identified as a compound which affects the processing of c-Ret by γ-secretase. For example, a compound which increases c-Ret cleavage product or decreases c-Ret undegraded product compared with control is evaluated as an accelerator for the c-Ret degradation activity of γ-secretase. On the other hand, a compound which decreases c-Ret cleavage product or increases c-Ret undegraded product compared with control is evaluated as an inhibitor for the c-Ret degradation activity of γ-secretase. The accelerator for the degradation activity of γ-secretase obtained by the method of the present invention is potentially useful for treatment of AD.

Analysis of c-Ret cleavage is performed by measuring an indicator of cleavage for one or both of the N-terminal fragment and C-terminal fragment of c-Ret.

As indicators of cleavage, the following antibodies may be used: anti-c-Ret antibodies; antibodies recognizing a c-Ret derivative (c-Ret undegraded product) before degradation by γ-secretase, produced as a result of non-cleavage of c-Ret; antibodies recognizing a c-Ret cleavage product produced as a result of cleavage of c-Ret; or antibodies recognizing the intracellular domain of c-Ret.

When c-Ret is tagged, a substance which binds to the tag (e.g., antibody) may be used to detect the indicator.

For example, when a tagged undegraded product of c-Ret polypeptide is to be detected, an hemagglutinin (HA) tag may be added to the C-terminus of c-Ret, followed by detection with an anti-HA tag antibody. In this case, it is possible to clarify the presence and concentration of a C-terminal fragment of c-Ret produced as c-Ret undegraded product, by detecting or quantitatively determining the HA tag. When c-Ret or tagged c-Ret is labeled, it is possible to detect its undegraded product by detecting or quantitatively determining the label.

On the other hand, when a cleavage product of c-Ret polypeptide is to be detected, the membrane fraction is purified from c-Ret-expressing cells; then, the purified membrane fraction is subjected to cleavage reaction by γ-secretase to thereby allow the c-Ret cleavage product to be released from the membrane fraction. When this reaction product is centrifuged, the c-Ret undegraded product is precipitated into the membrane fraction. Thus, the membrane fraction and other fractions can be separated. By detecting the released fragment, the c-Ret cleavage product can be detected. For this detection, an antibody recognizing the intracellular domain of c-Ret is used when endogenous c-Ret is used. When exogenous recombinant c-Ret is used, a cDNA encoding c-Ret with a tag sequence added to its C-terminus is used to express the recombinant c-Ret. Then, the c-Ret cleavage product is detected using an antibody that recognizes the tag. The tag is not particularly limited. For example, HA, Myc, FLAG or the like may be used.

The antibody to c-Ret is not particularly limited as long as the antibody recognizes c-Ret. Preferably, an antibody which recognizes the intracellular domain of c-Ret is used.

Those skilled in the art could prepare an antibody which recognizes c-Ret by immunizing an animal with an immunogen (antigen) and following the conventional, general procedures for preparing monoclonal antibodies. As the immunogen for example, c-Ret or a fragment thereof, or a fusion protein prepared by adding a tag or label to c-Ret or a fragment thereof may be used.

For example, a non-human mammal is immunized with the immunogen alone or, if necessary, together with Freund's adjuvant. Polyclonal antibodies may be obtained from the serum collected from the immunized animal. Monoclonal antibodies may be obtained by fusing antibody producing cells from the immunized animal with myeloma cells without autoantibody producing ability to prepare fusion cells (hybridomas), cloning the hybridomas, and selecting those clones which produce a monoclonal antibody showing specific affinity to the antigen used for immunizing the animal. The preparation of monoclonal antibodies from hybridomas may be performed in vitro. Alternatively, the preparation may be performed in vivo in a non-human mammal, preferably mouse or rat, more preferably in the abdominal dropsy in mouse. Monoclonal antibodies may be isolated from the resultant culture supernatant or the abdominal dropsy of the mammal. The isolation and purification of monoclonal antibodies may be performed by subjecting the above-mentioned culture supernatant or abdominal dropsy to methods such as saturated ammonium sulfate precipitation, euglobulin precipitation, caproic acid method, caprilic acid method, ion exchange chromatography (DEAE, DE52, etc.), or affinity column chromatography using anti-immunoglobulin column or protein A column. The monoclonal antibody include those monoclonal antibodies consisting of heavy chains and/or light chains having the amino acid sequences which have deletion, substitution or addition of one or several amino acids in the heavy chains and/or light chains constituting the initial antibody.

By the procedures described above, it is possible to incubate γ-secretase and c-Ret in the presence or absence of a candidate compound and to evaluate the cleavage of c-Ret by γ-secretase.

In another embodiment of the present invention, it is possible to evaluate whether or not a candidate compound affects the processing of APP and/or Notch, in parallel with, simultaneously with, or before or after the above-described method of the present invention. For example, a step of measuring the cleavage of APP or a polypeptide containing an APP cleavage site by γ-secretase (i.e., polypeptide containing APP γ-secretase cleavage site) may be included in parallel with, simultaneously with, or before or after the above-described method of the present invention. Further, a step of measuring the cleavage of Notch or a polypeptide containing a Notch cleavage site by γ-secretase (i.e., polypeptide containing Notch γ-secretase cleavage site) may be included.

By including such steps, it is possible to evaluate whether or not a candidate compound selectively acts on the processing of c-Ret compared to the processing of APP and/or Notch. The expression “selectively act on” used herein means to have more inhibitory effect or accelerating effect upon the cleavage of substrate c-Ret by γ-secretase than upon the cleavage of other substrate(s). Specifically, according to this embodiment of the present invention, it is possible to identify a compound which selectively acts only on the processing of c-Ret; a compound which selectively acts only on the processing of APP; a compound which selectively acts only on the processing of Notch; or a compound which selectively acts on the processing of APP and c-Ret. This means that, with the method of the present invention, it is possible to obtain compounds which selectively inhibit or activate the γ-secretase's cleavage activity on c-Ret in addition to γ-secretase's known substrates such as APP or Notch. Therefore, it is possible to obtain compounds which selectively inhibit or activate the cleavage function of γ-secretase on a specific substrate; compounds with increased selectivity on the substrate of γ-secretase can be obtained.

As a candidate compound in drug development, a compound which acts in the same manner as metabolic activity in healthy animals in vivo or a compound which regulates the metabolic activity is preferable. A preferable example of such a candidate compound is a compound which inhibits the production of Aβ42 by inhibiting the APP cleavage activity of γ-secretase, does not inhibit the c-Ret cleavage activity of γ-secretase, and does not inhibit the Notch cleavage activity of γ-secretase. Another preferable example is a compound which inhibits the production of Aβ42 by accelerating the production of Aβ40 through acceleration of the APP cleavage activity of γ-secretase; accelerates the degradation of c-Ret by accelerating the c-Ret cleavage activity of γ-secretase; and does not inhibit the Notch cleavage activity of γ-secretase.

As methods for measuring the cleavage by γ-secretase of APP, Notch or a polypeptide containing an APP or Notch γ-secretase cleavage site, assay methods known to those skilled in the art may be applicable (Song et al. PNAS 1999 96 6959-6963; Moehlmann et al. PNAS 2002 99 8025-8030). For example, a first biological composition containing γ-secretase or a biologically active fragment thereof may be contacted with a biological composition containing APP or a polypeptide containing an APP γ-secretase cleavage site or a biological composition containing Notch or a polypeptide containing a Notch γ-secretase cleavage site in the presence and absence of a candidate compound, and then the cleavage of the APP or the polypeptide containing an APP γ-secretase cleavage site or the cleavage of the Notch or the polypeptide containing a Notch γ-secretase cleavage site. This measurement may be performed by measuring the cleavage product from the APP or the polypeptide containing an APP γ-secretase cleavage site or the cleavage product from the Notch or the polypeptide containing a Notch γ-secretase cleavage site. As one example of the cleavage product from APP or a polypeptide containing an APP γ-secretase cleavage site, Aβ may be given. The quantity of Aβ may be measured, and changes in the quantity between the presence and absence of the candidate compound may be compared. Alternatively, the degree of cleavage and the quantity of cleavage product may be measured by using a known antibody which recognizes the cleavage product from APP or a polypeptide containing an APP γ-secretase cleavage site or the cleavage product from Notch or a polypeptide containing a Notch γ-secretase cleavage site. As the antibody which recognizes the cleavage product from APP or a polypeptide containing an APP γ-secretase cleavage site, commercial antibodies (Sigma or Chemicon) may be used. The measurement may be performed, for example, by Western blotting. As the antibody which recognizes the cleavage product from Notch or a polypeptide containing a Notch γ-secretase cleavage site, commercial antibodies (Santa Cruz) may be used. The measurement may be performed, for example, by Western blotting.

The method of the present invention also includes a high-throughput screening (HTS) known to those skilled in the art, which tests a large number of compounds simultaneously (U.S. Pat. Nos. 5,876,946 and 5,902,732; Jayawickreme and Kost. Cliff. Opin. Biotechnol. (1997), Vol. 8, pp. pages 629-634; Houston and Banks. Curt Opin. Biotechnol. (1997), Vol. 8, pp. 734-740).

The method of the present invention also includes the use of known model animals It is possible to analyze the in vivo effect of a compound selected by the in vitro method of the present invention by using, for example, non-human models for APP processing and/or AD. As non-human models for AD, APP transgenic non-human animal models are well-known in the art. As a method of using a model animal, Tg2576 mouse (J. Neurosci. 21(2), 372-381, 2001; J. Clin. Invest., 112, 440-449, 2003) may be used, and the following evaluations may be made after administering to Tg2576 mouse a known γ-secretase inhibitor DAPT, (2S)-2-{[(3,5-difluorophenyl)acetyl]amino}-N-[(3S)-1-methyl-2-oxo-5-phenyl-2,3-dihydro-1H-1,4-benzodiazepin-3-yl]propanamide (Alexis Biochemicals) or a compound of the present invention: evaluation by a method of measuring the Aβ quantities in the brain, cerebrospinal fluid and serum of the mouse (J. Pharmacol. Exp. Ther. 305, 864-871, 2003); pathological examination of changes in the brain (e.g., changes in Aβ yield, the degree of cerebral atrophy, etc.) resulting from changes in γ-secretase activity; and evaluation of the survival ratio, momentum or food consumption of the mouse.

The pharmaceutical composition comprising the compound identified by the method of the present invention, preferably the AD therapeutic of the present invention, may be administered to patients in various forms through an oral or parenteral (e.g., intravenous injection, muscle injection, subcutaneous administration, rectal administration or transdermal administration) route. Therefore, the pharmaceutical composition comprising the compound of the present invention may be formulated into various preparations using a pharmacologically acceptable carrier by a conventional method depending on the administration route, though the pharmaceutical composition may be used alone.

Preferred dosage forms include oral preparations such as tablets, powders, subtle granules, granules, coated tablets, capsules, syrups and troches; and parenteral preparations such as inhalants, suppositories, injections (including drops), ointments, eye drops, ophthalmic ointments, nasal drops, ear drops, cataplasms, and lotions and liposomes.

Examples of carriers used in the formulation include conventionally used fillers, binders, disintegrants, lubricants, coloring agents and flavoring agents, as well as stabilizers, emulsifiers, absorbefacients, surfactants, pH adjusting agents, antiseptics, antioxidants, expanders, wetting agents, surface activators, dispersing agents, buffers, preservatives, dissolution aids and analgesic agents according to necessity. They can be formulated according to a conventional procedure using components commonly used as raw materials for pharmaceutical preparations. Examples of nontoxic these components which may be used in the present invention include animal and vegetable oils such as soybean oil, beef tallow and synthetic glycerides; hydrocarbons such as liquid paraffins, squalane and solid paraffins; ester oils such as octyldodecyl myristate and isopropyl myristate; higher alcohols such as cetostearyl alcohol and behenyl alcohol; silicone resins; silicone oils; surfactants such as polyoxyethylene fatty acid esters, sorbitan fatty acid esters, glycerin fatty acid esters, polyoxyethylene sorbitan fatty acid esters, polyoxyethylene hydrogenated castor oils and polyoxyethylene-polyoxypropylene block copolymers; water-soluble polymers such as hydroxyethyl cellulose, polyacrylic acids, carboxyvinyl polymers, polyethylene glycol, polyvinylpyrrolidone and methylcellulose; lower alcohols such as ethanol and isopropanol; polyhydric alcohols (polyols) such as glycerol, propylene glycol, dipropylene glycol, sorbitol and polyethylene glycol; sugars such as glucose and sucrose; inorganic powders such as silicic anhydride, magnesium aluminium silicate and aluminium silicate; inorganic salts such as sodium chloride and sodium phosphate; and purified water.

The fillers include, for example, lactose, fructose, corn starch, white sugar, glucose, mannitol, sorbitol, crystalline cellulose and silicon dioxide. The binders include, for example, polyvinyl alcohol, polyvinyl ether, methylcellulose, ethylcellulose, gum arabic, gum tragacanth, gelatin, shellac, hydroxypropyl methylcellulose, hydroxypropyl cellulose, polyvinylpyrrolidone, polypropylene glycol-polyoxyethylene block polymers and meglumine. The disintegrants include, for example, starch, agar, gelatin powder, crystalline cellulose, calcium carbonate, sodium hydrogencarbonate, calcium citrate, dextrin, pectin and carboxymethylcellulose calcium. The lubricants include, for example, magnesium stearate, talc, polyethylene glycol, silica and hardened vegetable oils. The coloring agents may be any coloring agents which are approved to be added to pharmaceutical preparations. The flavoring agents include, for example, cocoa powder, menthol, aromatic powder, peppermint oil, camphol and cinnamon powder. The above-listed components may be in the form of a salt or solvate thereof.

The oral preparation is produced by mixing the compound of the present invention with a filler, and if necessary, a binder, disintegrant, lubricant, coloring agent, flavoring agent, etc. and formulating the mixture according to conventional procedures into, for example, a powder, subtle granules, granules, tablet, coated tablet, capsules or the like. Resultant tablets and granules can be appropriately coated with, for example, sugar according to necessity. The syrups and injection preparations can be prepared according to conventional procedures by adding a pH adjusting agent, solubilizer, and isotonizing agent, and if necessary, a dissolution aid, stabilizer, etc. The external preparations can be produced according to conventional procedures not specifically limited. Base materials which may be used in the present invention include various raw materials conventionally used in pharmaceutical preparations, quasi drugs and cosmetics. Such raw materials include, for example, animal and vegetable oils, mineral oils, ester oils, waxes, higher alcohols, fatty acids, silicone oils, surfactants, phospholipids, alcohols, polyhydric alcohols, water-soluble polymers, clay minerals and purified water. If necessary, pH adjusting agents, antioxidants, chelating agents, antiseptics and antimolds, coloring agents, flavors, or the like can be added. In addition, components such as blood-flow accelerators, bactericides, anti-inflammatory agents, cell activators, vitamins, amino acids, humectants, keratolytic agents or the like be added according to necessity. The ratio of the active ingredient to carriers may vary from 1 to 90% by weight. When the compounds used in the present invention, the peptides used in the present invention or the polynucleotides used in the present invention are used in the above-described treatment, it is preferable to use those compounds, peptides or polynucleotides purified to 90% or more, preferably 95% or more, more preferably 98% or more, still more preferably 99% or more.

The effective dose of the pharmaceutical composition comprising the compound of the present invention varies depending on the severity of symptom, the age, sex and body weight of the patient, administration mode, type of the salt, specific type of the disease and other factors. Generally, the pharmaceutical composition may be administered to an adult (body weight: 60 kg) in one to several divided doses at a daily dose of about 30 μg to about 10 g, preferably 100 μg to 5 g, and more preferably 100 μg to 100 mg for oral administration; or at a daily dose of about 30 μg to about 1 g, preferably 100 μg to 500 mg, and more preferably 100 μg to 30 mg for injection administration. Considering that efficacy varies depending on the administration route, the required dose is expected to vary widely. For example, it is expected that oral administration requires a higher dose than intravenous injection. When administered to children, the dose may be smaller than the dose for adults. These variations in the dose level can be adjusted by standard empirical optimization procedures which are well understood in the industry.

The term “treatment” is used herein to generally mean obtaining a desired pharmacological and/or physiological effect. The effect may be prophylactic in terms of completely or partially preventing a disease and/or a symptom and may be therapeutic in terms of partially or completely curing a disease and/or an adverse effect attributed to the disease. The term “treatment” as used herein covers any treatment of a disease in a patient, preferably a human, and includes at least one treatment selected from the following (a) to (c):

(a) preventing a disease or a symptom from occurring in a patient who may be predisposed to the disease but has not yet been diagnosed as having it;
(b) inhibiting a disease symptom, i.e. preventing or delaying its progress; or
(c) relieving a disease symptom, i.e. causing regression or elimination of the disease or symptom, or causing reversal of the progress of the disease.

For example, as clinical symptoms of AD, progressive disorientation, memory loss and aphasia are enumerated. Finally, disablement, speech loss and akinesia occur. Pathological signs of AD include neurofibrillary tangles, senile plaques and amyloid angiopathy. To prevent the progress of AD is interpreted to mean to prevent the onset or further progress of the clinical symptoms and/or pathological signs of AD. For example, in patients who do not have the clinical symptoms or pathological signs of AD, it is possible to prevent the progress of clinical symptoms or pathological signs. In patients suffering from mild AD, it is possible to prevent the development of more severe AD forms. To delay the progress of AD is interpreted to mean to delay the point of onset of AD-related symptoms and/or pathological signs, or to reduce the speed of progress of AD that is determined by the speed of progress of clinical symptoms and pathological signs. To reverse the progress of AD is interpreted to mean to relieve the severity of AD symptoms, i.e., to change the severity of AD conditions of patients from severe to mild. At that time, the change to mild is indicated by decrease of clinical symptoms or pathological signs.

Diagnosis of AD in patients may be performed by various known methods. Typically, AD is diagnosed by combining clinical and pathological assessments. For example, the progress or severity of AD may be judged using Mini Mental State Examination (MMSE) (Mohs et al. (1996) Int Psychogeriatr 8: 195-203), Alzheimer's Disease Assessment Scale-Cognitive Subscale (ADAS-cog) (Galasko et al., (1997) Alzheimer Dis Assoc Disord, 11 suppl 2: S33-9), Alzheimer's Disease Cooperative Study-Activities of Daily Living (ADCS-ADL) (McKhann et al., (1984) Neurology 34: 939-944) and Criteria of National Institute of Neurologic Communicative Disorders and Stroke-Alzheimer's Disease and Related Disorders Association (NINCDS-ADRDA) (Folstein et al., (1975) J Psychiatr Res 12: 189-198; McKhann et al., (1984) Neurology 34: 939-944). Further, methods which evaluate various regions of the brain and enable the estimation of frequency of senile plaques or neurofibrillary tangle may be used (Braak et al., (1991) Acta Neuropathol 82: 239-259; Khachaturian (1985) Arch Neuro 42: 1097-1105; Mirra et al., (1991) Neurology 41: 479-486; and Mirra et al., (1993) Arch Pathol Lab Med 117: 132-144).

The present invention provides a test kit for measuring the processing of c-Ret by γ-secretase as a test kit for use in the method of the present invention.

The kit of the present invention comprises at least γ-secretase or a biological composition containing γ-secretase, and a biological composition containing c-Ret. The kit may further comprise a substrate for γ-secretase other than c-Ret (e.g., APP and/or Notch) or a biological composition containing such a substrate. Preferably, the kit comprises a plurality of substrates for γ-secretase. It is preferred that the kit comprises APP and/or Notch in addition to c-Ret.

Further, the test kit of the present invention may comprise tools, reagents, instructions, and so forth which are used in technologies for detecting the processing of c-Ret by γ-secretase. Examples of technologies for detecting the processing of c-Ret by γ-secretase include immunoblotting and Western blotting. Examples of tools used in these technologies include reaction vessels and blotting membranes; and examples of reagents used in these technologies include buffers, culture broths and anti-c-Ret antibodies.

With the kit of the present invention, it is possible to measure the processing of c-Ret by γ-secretase. As a result, it becomes possible to evaluate whether or not a candidate compound affects the processing of c-Ret by γ-secretase. Therefore, this kit can serve as a test kit for screening for compounds which affect the processing of c-Ret by γ-secretase. The test kit of the present invention includes a test kit for screening for compounds which affect the processing of c-Ret by γ-secretase, the kit having the same constitution as that of the test kit for measuring the processing of c-Ret by γ-secretase.

Further, the present invention includes the use of the above-described kit in measuring the processing of c-Ret, or in methods of screening or testing γ-secretase inhibitors.

### EXAMPLES

Hereinbelow, the present invention will be described in more detail with reference to the following Examples and Preparation Examples. However, the present invention is not limited to these Examples, which are provided only for the purpose of full disclosure of the present invention to those skilled in the art. It is not meant or even implied that the experiments described herein are all or only one experiment actually carried out. Although efforts have been made to guarantee the accuracy of the numerical values used herein (e.g., volume, temperature, concentration, etc.), experimental errors and deviations are considered to some extent. Thus, such values may be changed within a range which does not depart from the scope of the present invention.

### Example 1

### Analysis of c-Ret Processing in c-Ret-Transfected 293/EBNA-1 Cell Strain

Whether or not c-Ret is a substrate for γ-secretase was evaluated using HEK293 cells expressing a gene encoding c-Ret with an HA tag added to its C-terminus, in the presence of a γ-secretase inhibitor. As the γ-secretase inhibitor, (2S)-2-{[(3,5-difluorophenyl)acetyl]amino}-N-[(3S)-1-methyl-2-oxo-5-phenyl-2,3-dihydro-1H-1,4-benzodiazepin-3-yl]propanamide (hereinafter, sometimes referred to as “Compound E”) (Alexis Biochemicals) was used.

### 1. Experimental Conditions and Methods

(1) Cloning of Rat c-Ret

RNA was purified from rat brain with TRIzol (Invitrogen), followed by synthesis of 1st strand cDNA with RNA PCR Kit (TaKaRa). c-Ret was amplified in two separate fragments of 1-767 by (positions from 1 to 767 in the nucleotide sequence as shown in SEQ ID NO: 9) and 1681 bp-stop (positions from 1681 to 3351 in the nucleotide sequence as shown in SEQ ID NO: 9). Specifically, c-Ret was amplified using 1 μl of the finally synthesized 1st strand cDNA product, the following primers and Pfu (Stratagene).

Primer 1—SalI site added:

(SEQ ID NO: 31)
GAGGTCGACGCCACCATGGCGAAAGCGAGGTCCGGCGC
Primer 2:
(SEQ ID NO: 32)
ACGGAGACAGTCCTGGGGGCAAA
Primer 3:
(SEQ ID NO: 33)
TCTCCTAGCACCAGGACCTGTCC

Primer 4—NotI site added:

(SEQ ID NO: 34)
GAGGCGGCCGCGCTATCAAATGTGTCCATTAATTT

PCR conditions were as follows: first reaction at 95° C. for 2 minutes; then 35 cycles of (at 95° C. for 45 seconds→at 60° C. for 45 seconds→at 72° C. for 6 minutes); then final reaction at 72° C. for 10 minutes. The PCR products were purified with Quiaquick PCR purification kit (QIAGEN). Then, the 1-767 by fragment was treated with restriction enzymes SalI (TaKaRa) and EcoRV (TaKaRa), and the 1681 bp-stop fragment was treated with EcoRV (TaKaRa) and NotI (TaKaRa). Subsequently, they were cloned into pBluescript (Stratagene), separately. The resultant pBluescripts were subjected to sequence analysis with a DNA sequencer (Applied Biosystems; 3130×1). The resultant pBluescript 1-767 by was treated with restriction enzymes EcoRV and NotI. Then, the 1681 bp-stop fragment was inserted thereto.

(2) Construction of a Gene Encoding Rat c-Ret with HA Tag Added to its C-Terminus and an Expression Vector

pcDNA (Invitrogen) was treated with restriction enzymes SalI (TaKaRa) and NotI (TaKaRa). c-Ret obtained by treating the pBluescript obtained in (1) above with restriction enzymes SalI (TaKaRa) and NotI (TaKaRa) was inserted into the resultant pcDNA to thereby prepare an expression vector. This expression vector was constructed so that an HA tag is added to the C-terminus of the incorporated c-Ret. The DNA sequence of the resultant c-Ret-HA was analyzed with a DNA sequencer (Applied Biosystems; 3130×1). The resultant DNA sequence is shown in SEQ ID NO: 13.

In the resultant DNA sequence, the nucleotide at position 89 was changed from g to a, compared with rat c-Ret (NM_{—}012643; SEQ ID NO: 9). (Hereinafter, this mutation is expressed as “g→a”. Other mutations will also be expressed in the same manner.) Besides, when compared with rat c-Ret (NM_{—}012643), the resultant DNA sequence had g→a mutation at position 270, c→g mutation at position 435, a→g mutation at position 504, t→g mutation at position 675, a→g mutation at position 684, t→g mutation at position 693, g→a mutation at position 1070, g→c mutation at position 1095, t→g mutation at position 1168, g→c mutation at position 1422, a→g mutation at position 1497, c→a mutation at position 1581, c→t mutation at position 1658, a→g mutation at position 1809, c→g mutation at position 1886, a→g mutation at position 2249, t→a mutation at position 2754, a→g mutation at position 2767, t→g mutation at position 2769, c→t mutation at position 2777, t→a mutation at position 2785, a→g mutation at position 2835, a→g mutation at position 2926 and t→c mutation at position 3195, with a deletion of tga at positions from 798 to 800. With respect to the relation of nucleotide positions when the DNA sequences of c-Ret-HA (SEQ ID NO: 13) and rat c-Ret (NM_{—}012643; SEQ ID NO: 9) are compared, the nucleotide positions of NM_{—}012643 (SEQ ID NO: 9) are regarded as reference. Therefore, in the nucleotide sequence of SEQ ID NO: 13, nucleotides at position 798 and thereafter are shifted by three nucleotides compared with the nucleotide sequence of SEQ ID NO: 9 because of the above-described deletion. For example, the position corresponding to position 1070 in SEQ ID NO: 9 is 1067 in SEQ ID NO: 13. Further, the unidentified nucleotides in NM_{—}012643 were identified as follows: position 1485 was c; position 1997 was c; position 2000 was a; position 2001 was c; position 2015 was c; position 2022 was c; position 2043 was g; position 2052 was c; position 2080 was a; and position 2735 was a. At the amino acid level, the sequence had C→Y mutation (mutation from C to Y) at position 30, R→K mutation at position 357, F→V mutation at position 390, T→I mutation at position 553, A→G mutation at position 629, N→K mutation at position 918, N→E mutation at position 923, S→F mutation at position 926, F→I mutation at position 929 and N→D mutation at position 976, with a deletion of D at position 266 because of the above-described tga deletion at positions 798-800. Further, the unidentified amino acid residues were identified as follows: position 495 was R; position 666 was A; position 667 was H; position 672 was A, position 674 was A; position 681 was P; position 684 was G; position 694 was T; and position 912 was K. The expression vector was prepared in large quantity with Endofree Plasmid Maxi Kit (QIAGEN). It should be noted here that when the amino acid sequence of c-Ret-HA (SEQ ID NO: 14) is compared with that of rat c-Ret (NM_{—}012643; SEQ ID NO: 10), the amino acid sequence from position 266 and thereafter of SEQ ID NO: 14 is shifted by one amino acid relative to the amino acid sequence as shown in SEQ ID NO: 10.

The 3′ terminal sequence (from g at 3355 to t at 3384) of the nucleotide sequence as shown in SEQ ID NO: 13 is a nucleotide sequence encoding the HA tag.

(3) Cells Expressing the Gene Encoding Rat c-Ret with HA Tag Added to its C-Terminus

293/EBNA-1 cell strain (Invitrogen) was cultured in 6-well dishes containing 10% FBS (Hyclone)/DMEM (Invitrogen) under 5% CO₂ at 37° C., followed by transfection thereinto of the gene encoding rat c-Ret with an HA tag added to its C-terminus using Lipofectamine 2000 (Invitrogen). After one day culture under the same conditions, 50 mM Compound E (Alexis Biochemicals) was added to the culture broth. Cells were cultured for another day under the same conditions. Then, the transfected HEK293 cells were collected with PBS (Sigma) and sonicated with a sonicator (Taitec VP-5S) to disrupt cells. Then, the quantity of protein was determined with Protein Assay Kit (BioRad). Samples (2 μg each) were taken from proteins obtained from Compound E-added cells and Compound E-not added cells, respectively, and subjected to SDS-PAGE, followed by Western blotting with an anti-HA antibody (Roche) (final concentration: 0.2 μg/ml).

### 2. Experimental Results

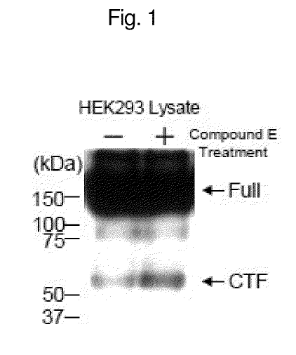

FIG. 1 shows the results.

In FIG. 1, the left lane represents the sample untreated with Compound E and the right lane represents the sample treated with Compound E. “Full” represents the full-length of c-Ret before degradation (about 150 kDa). CTF (C terminal fragment) represents a region spanning from the transmembrane domain of c-Ret to the C terminal domain. When Compound E (γ-secretase inhibitor) was added, a band around 50 kDa (CTF) was accumulated specifically as a c-Ret undegraded product. Since this band is equal in size to the region spanning from the transmembrane domain of c-Ret to its C-terminal domain, it has become clear that c-Ret is cleaved by γ-secretase in HEK293 cells.

The technical terms used herein are used only for the purpose of illustrating a specific embodiment and not intended to limit the embodiment.

Unless otherwise specifically defined, all technical terms and scientific terms used herein have the same meaning as generally understood by those skilled in the art. Although any methods and materials similar to or equivalent to those described herein may be used in the practice or test of the present invention, those skilled in the art can consult the description provided herein, for preferable methods and materials.

All publications cited herein are incorporated herein by reference in their entirety for the purpose of describing and disclosing, for example, the cell systems, constructs and methods described in the publications that are used in connection with the present invention, or incorporated herein as references with respect to the disclosure of the compound identification method, screening method and methodologies therefor, and composition of the present invention; such publications may be used for the practice of the present invention.

1. A method of screening for compounds which affect the processing of c-Ret by γ-secretase, comprising the following steps:
(i) contacting a first biological composition containing γ-secretase or a biologically active fragment thereof with a second biological composition containing c-Ret in the presence and absence of a candidate compound;
(ii) measuring the cleavage of the c-Ret in the presence and absence of the candidate compound;
(iii) selecting those candidate compounds which affect the cleavage of the c-Ret by γ-secretase; and
(iv) identifying the candidate compounds selected in step (iii) as compounds which affect the processing of c-Ret by γ-secretase.

2. The method of claim 1, further comprising evaluating a candidate compound as an inhibitor of c-Ret degradation activity of γ-secretase when c-Ret undegraded product in the presence of said candidate compound is increased relative to c-Ret undegraded product in the absence of said candidate compound in the cleavage of c-Ret measured in step (ii).

3. The method of claim 1, further comprising evaluating a candidate compound as an accelerator of c-Ret degradation activity of γ-secretase when c-Ret undegraded product in the presence of said candidate compound is decreased relative to c-Ret undegraded product in the absence of said candidate compound in the cleavage of c-Ret measured in step (ii).

4. The method of claim 1, further comprising evaluating a candidate compound as an accelerator of c-Ret degradation activity of γ-secretase when c-Ret cleavage product in the presence of said candidate compound is increased relative to c-Ret cleavage product in the absence of said candidate compound in the cleavage of c-Ret measured in step (ii).

5. The method of claim 1, further comprising evaluating a candidate compound as an inhibitor of c-Ret degradation activity of γ-secretase when c-Ret cleavage product in the presence of said candidate compound is decreased relative to c-Ret cleavage product in the absence of said candidate compound in the cleavage of c-Ret measured in step (ii).

6. The method of claim 1, further comprising measuring the cleavage of APP or a polypeptide comprising an APP γ-secretase cleavage site.

7. The method of claim 1, further comprising measuring the cleavage of Notch or a polypeptide comprising a Notch γ-secretase cleavage site.

8. A test kit for measuring the processing of c-Ret by γ-secretase, comprising a first biological composition containing γ-secretase or a biologically active fragment thereof and a second biological composition containing c-Ret.


SEQUENCE LISTING (34 sequences)

SEQ ID NO: 1 - DNA, 3345 bp, Homo sapiens
  CDS (1)..(3342)
atggcgaaggcgacgtccggtgccgcggggctgcgtctgctgttgctgctgctgctgccgctgctaggcaaagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggcatgtcagacccgaactggcctggagagagtcctgtaccactcacgagagctgatggcactaacactgggtttccaagatatccaaatgatagtgtatatgctaactggatgctttcaccctcagcggcaaaattaatggacacgtttgatagttaa

SEQ ID NO: 2 - protein, 1114 aa, Homo sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 3 - DNA, 2583 bp, Homo sapiens
  CDS (1)..(2580)
atggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcgttcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggcatgtcagacccgaactggcctggagagagtcctgtaccactcacgagagctgatggcactaacactgggtttccaagatatccaaatgatagtgtatatgctaactggatgctttcaccctcagcggcaaaattaatggacacgtttgatagttaa

SEQ ID NO: 4 - protein, 860 aa, Homo sapiens
MVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 5 - DNA, 3219 bp, Homo sapiens
  CDS (1)..(3216)
atggcgaaggcgacgtccggtgccgcggggctgcgtctgctgttgctgctgctgctgccgctgctaggcaaagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 6 - protein, 1072 aa, Homo sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 7 - DNA, 3219 bp, Homo sapiens
  CDS (1)..(3216)
atggcgaaggcgacgtccggtgccgcggggctgcgtctgctgttgctgctgctgctgccgctgctaggcaaagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccagtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcgtacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtactgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 8 - protein, 1072 aa, Homo sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSSARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYCLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 9 - DNA, 3351 bp, Rattus norvegicus
  CDS (1)..(3348)
  misc_feature (1485)..(1485): n is a, c, g, or t
atggcgaaagcgaggtccggcgccgcagggctggggctgaagctgtttttgctgctgccgctactgggagaagccccgctgggtctctgcttctcaagggatgcttactgggagaggctgtatgtggaccagccagctggcacacctctgctctatgtccatgccctacgggatgcccctggagaagtgcccagcttccgcctgggccagtatctctatggcgtctaccgcacgcgtctgcatgagaatgactggatccacatcgatgcgggcactggcctcctctacctcaatcagagcctggaccatagttcctgggagcagctcagcatccgaaatggcggcttccccttgctcaccgtcttcctccaggtcttcctggggtccacagcccagagagagggagagtgtcattggccaggctgtgcccgtgtctacttctccttcatcaacgacaccttcccaaattgtagctccttcaaagcccgggatctctgcaccccagagacgggtgtgtccttccgcatcagggagaacaggccccctggcaccttctaccagttccgcatgctacctgtgcagttcctttgtcctaacatcagtgtgaagtacaaactcttagaaggggacggtctgcccttccgttgtgaccccgactgtctggaggtgagcactcggtgggcactggatcgtgagcttcaggagaagtatgtgctggaggctgagtgcgcagtggcaggccctggagccaacaaggagaaggtggccgtgtccttcccggtgacggtgtatgatgatgaagacgactccccgcccaccttctccggaggtgtgggcaccgccagtgctgtggtggagtttaagcggaaggagggcactgtggtagccactctgcaggtgtttgatgcagatgtggtgccagcatctggggagctggtgaggcggtacacaagcacactactctcaggggattcctgggcccagcagaccttccgggtggagcacacacccaacgagaccttggtccagtccaacaacaactccgtgcgggcaaccatgcacaattacaggctggttctcaacaggagcctgtcgatctcagagagccgagtcctgcagctagtagtcctggtcaatgactcagacttccaggggcctgggtcaggttttctcttcctccatttcaacgtgtctgtgctgcctgtcaccctgaacctacccatggcctactccttcccagtgaataggagagcccgccgttatgcccagattgggaaagtttgcgtggagaactgccaggagttcagcggtgtctccatccagtacaagctgcagccctccagcaccaactgcagtgccctaggtgtggtcacctcaacagaagacacctcagggaccctatatgtaaatgacacggaggcgctgcggcgacctgagtgtaccgagcttcagtacacagtggtagccactgaccggcagacccgnaggcagacccaagcttcgttagtcgtcacagtggaggggacatacattgcagaagaagtgggctgccccaagtcctgtgcagtaaacaagaggcgccctgagtgtgaggagtgtggtggcctgggttctccaactggcagatgtgagtggcgtcagggagatggtaaagggaccaccaggaacttctccacctgttctcctagcaccaggacctgtcctgatggccactgtgatgctctggagagccgggatatcaacatttgcccccaggactgtctccgtggccccattgttggcgggcatgagcgaggggagcgccagggaattaaagccggctatggcatctgcaactgtttccctgatgagaagaagtgcttctgcgagccagaggacagccaggccccattgtgcgatgagctgtgccgtacggtcatcacagccgctgtcctcttctccttcataatctctgtcctgctgtccaccttctgcatccaccgctaccacaagcatgngcnnaagccacccatcgngtcagcngaaatgaccttctgccggccngcccagggnttcccaatcagctattcttcctcgggcncccgccggccctcactggactccatggagaaccaggtctctgtggactctttcaagatcccggaggatccgaagtgggaatttcctcggaagaacttagttcttgggaaaaccctgggagaaggcgagtttggaaaagtagtcaaggccacagccttccgtctgaaagaccgggcaggatacaccacagtggctgtgaaaatgctgaaagaaaacgcctcccagagtgaactacgagacctgctctctgagttcaaccttctgaaacaagtcaaccatccacatgtcatcaagttgtacggggcttgcagccaggatgggccacttcttctcattgtggagtatgcaaagtatggatccctgcgggggttcctgcgggacagccgcaagatcgggcctgcctatgtgagcagtggaggcagccgcaattccagctccctggaccacccagacgaaagggtgctgaccatgggcgacctcatctccttcgcctggcagatctcgaggggtatgcagtacttggctgaaatgaagctcgtacatcgagacttagctgccagaaacatcttggtggcagagggacggaagatgaagatctctgactttgggctgtcccgagatgtttatgaagaagattcctatgtgaagaaaagcangggccggattcccgtcaattggatggcaatcaattctctctccgatcacttctataccactcaaagtgatgtgtggtcctttggagtgctgctatgggaaattgtgaccctgggaggcaacccctaccctggaattcctcctgaacgactcttcaaccttctgaagacaggccacaggatggagaggccaaacaactgcagcgaggaaatgtaccgcctgatgctgcagtgctggaagcaggagccggacaagaggccagtatttgctgacatcagcaaggatctggagaagatgatggtcaaaagcagagactacttggacctggctgcatccaccccttcggactcactgctctatgacgatgggctctcggaagaggagacgcccctggtggactgtaacagtgctcccctcccgcgctccctcccttccacatggattgaaaacaaactctatggtatgtcagacccgaactggcctggagagagtcctgtaccactcacgagagccgatggcactagcactgggttcccaagatatgcaaatgatagtgtatatgctaactggatggtttcaccctcagcggcaaaattaatggacacatttgatagctaa

SEQ ID NO: 10 - protein, 1116 aa, Rattus norvegicus
  misc_feature (666)..(666): The ′Xaa′ at location 666 stands for Glu, Gly, Ala, or Val.
MAKARSGAAGLGLKLFLLLPLLGEAPLGLCFSRDAYWERLYVDQPAGTPLLYVHALRDAPGEVPSFRLGQYLYGVYRTRLHENDWIHIDAGTGLLYLNQSLDHSSWEQLSIRNGGFPLLTVFLQVFLGSTAQREGECHWPGCARVYFSFINDTFPNCSSFKARDLCTPETGVSFRIRENRPPGTFYQFRMLPVQFLCPNISVKYKLLEGDGLPFRCDPDCLEVSTRWALDRELQEKYVLEAECAVAGPGANKEKVAVSFPVTVYDDEDDSPPTFSGGVGTASAVVEFKRKEGTVVATLQVFDADVVPASGELVRRYTSTLLSGDSWAQQTFRVEHTPNETLVQSNNNSVRATMHNYRLVLNRSLSISESRVLQLVVLVNDSDFQGPGSGFLFLHFNVSVLPVTLNLPMAYSFPVNRRARRYAQIGKVCVENCQEFSGVSIQYKLQPSSTNCSALGVVTSTEDTSGTLYVNDTEALRRPECTELQYTVVATDRQTRRQTQASLVVTVEGTYIAEEVGCPKSCAVNKRRPECEECGGLGSPTGRCEWRQGDGKGTTRNFSTCSPSTRTCPDGHCDALESRDINICPQDCLRGPIVGGHERGERQGIKAGYGICNCFPDEKKCFCEPEDSQAPLCDELCRTVITAAVLFSFIISVLLSTFCIHRYHKHXXKPPIXSAEMTFCRPAQGFPISYSSSGXRRPSLDSMENQVSVDSFKIPEDPKWEFPRKNLVLGKTLGEGEFGKVVKATAFRLKDRAGYTTVAVKMLKENASQSELRDLLSEFNLLKQVNHPHVIKLYGACSQDGPLLLIVEYAKYGSLRGFLRDSRKIGPAYVSSGGSRNSSSLDHPDERVLTMGDLISFAWQISRGMQYLAEMKLVHRDLAARNILVAEGRKMKISDFGLSRDVYEEDSYVKKSXGRIPVNWMAINSLSDHFYTTQSDVWSFGVLLWEIVTLGGNPYPGIPPERLFNLLKTGHRMERPNNCSEEMYRLMLQCWKQEPDKRPVFADISKDLEKMMVKSRDYLDLAASTPSDSLLYDDGLSEEETPLVDCNSAPLPRSLPSTWIENKLYGMSDPNWPGESPVPLTRADGTSTGFPRYANDSVYANWMVSPSAAKLMDTFDS

SEQ ID NO: 11 - DNA, 3225 bp, Rattus norvegicus
  CDS (1)..(3222)
  misc_feature (1485)..(1485): n is a, c, g, or t
atggcgaaagcgaggtccggcgccgcagggctggggctgaagctgtttttgctgctgccgctactgggagaagccccgctgggtctctgcttctcaagggatgcttactgggagaggctgtatgtggaccagccagctggcacacctctgctctatgtccatgccctacgggatgcccctggagaagtgcccagcttccgcctgggccagtatctctatggcgtctaccgcacgcgtctgcatgagaatgactggatccacatcgatgcgggcactggcctcctctacctcaatcagagcctggaccatagttcctgggagcagctcagcatccgaaatggcggcttccccttgctcaccgtcttcctccaggtcttcctggggtccacagcccagagagagggagagtgtcattggccaggctgtgcccgtgtctacttctccttcatcaacgacaccttcccaaattgtagctccttcaaagcccgggatctctgcaccccagagacgggtgtgtccttccgcatcagggagaacaggccccctggcaccttctaccagttccgcatgctacctgtgcagttcctttgtcctaacatcagtgtgaagtacaaactcttagaaggggacggtctgcccttccgttgtgaccccgactgtctggaggtgagcactcggtgggcactggatcgtgagcttcaggagaagtatgtgctggaggctgagtgcgcagtggcaggccctggagccaacaaggagaaggtggccgtgtccttcccggtgacggtgtatgatgatgaagacgactccccgcccaccttctccggaggtgtgggcaccgccagtgctgtggtggagtttaagcggaaggagggcactgtggtagccactctgcaggtgtttgatgcagatgtggtgccagcatctggggagctggtgaggcggtacacaagcacactactctcaggggattcctgggcccagcagaccttccgggtggagcacacacccaacgagaccttggtccagtccaacaacaactccgtgcgggcaaccatgcacaattacaggctggttctcaacaggagcctgtcgatctcagagagccgagtcctgcagctagtagtcctggtcaatgactcagacttccaggggcctgggtcaggttttctcttcctccatttcaacgtgtctgtgctgcctgtcaccctgaacctacccatggcctactccttcccagtgaataggagagcccgccgttatgcccagattgggaaagtttgcgtggagaactgccaggagttcagcggtgtctccatccagtacaagctgcagccctccagcaccaactgcagtgccctaggtgtggtcacctcaacagaagacacctcagggaccctatatgtaaatgacacggaggcgctgcggcgacctgagtgtaccgagcttcagtacacagtggtagccactgaccggcagacccgnaggcagacccaagcttcgttagtcgtcacagtggaggggacatacattgcagaagaagtgggctgccccaagtcctgtgcagtaaacaagaggcgccctgagtgtgaggagtgtggtggcctgggttctccaactggcagatgtgagtggcgtcagggagatggtaaagggaccaccaggaacttctccacctgttctcctagcaccaggacctgtcctgatggccactgtgatgctctggagagccgggatatcaacatttgcccccaggactgtctccgtggccccattgttggcgggcatgagcgaggggagcgccagggaattaaagccggctatggcatctgcaactgtttccctgatgagaagaagtgcttctgcgagccagaggacagccaggccccattgtgcgatgagctgtgccgtacggtcatcacagccgctgtcctcttctccttcataatctctgtcctgctgtccaccttctgcatccaccgctaccacaagcatgngcnnaagccacccatcgngtcagcngaaatgaccttctgccggccngcccagggnttcccaatcagctattcttcctcgggcncccgccggccctcactggactccatggagaaccaggtctctgtggactctttcaagatcccggaggatccgaagtgggaatttcctcggaagaacttagttcttgggaaaaccctgggagaaggcgagtttggaaaagtagtcaaggccacagccttccgtctgaaagaccgggcaggatacaccacagtggctgtgaaaatgctgaaagaaaacgcctcccagagtgaactacgagacctgctctctgagttcaaccttctgaaacaagtcaaccatccacatgtcatcaagttgtacggggcttgcagccaggatgggccacttcttctcattgtggagtatgcaaagtatggatccctgcgggggttcctgcgggacagccgcaagatcgggcctgcctatgtgagcagtggaggcagccgcaattccagctccctggaccacccagacgaaagggtgctgaccatgggcgacctcatctccttcgcctggcagatctcgaggggtatgcagtacttggctgaaatgaagctcgtacatcgagacttagctgccagaaacatcttggtggcagagggacggaagatgaagatctctgactttgggctgtcccgagatgtttatgaagaagattcctatgtgaagaaaagcangggccggattcccgtcaattggatggcaatcaattctctctccgatcacttctataccactcaaagtgatgtgtggtcctttggagtgctgctatgggaaattgtgaccctgggaggcaacccctaccctggaattcctcctgaacgactcttcaaccttctgaagacaggccacaggatggagaggccaaacaactgcagcgaggaaatgtaccgcctgatgctgcagtgctggaagcaggagccggacaagaggccagtatttgctgacatcagcaaggatctggagaagatgatggtcaaaagcagagactacttggacctggctgcatccaccccttcggactcactgctctatgacgatgggctctcggaagaggagacgcccctggtggactgtaacagtgctcccctcccgcgctccctcccttccacatggattgaaaacaaactctatggtagaatttcacatgcatttactagattctag

SEQ ID NO: 12 - protein, 1074 aa, Rattus norvegicus
  misc_feature (666)..(666): The ′Xaa′ at location 666 stands for Glu, Gly, Ala, or Val.
MAKARSGAAGLGLKLFLLLPLLGEAPLGLCFSRDAYWERLYVDQPAGTPLLYVHALRDAPGEVPSFRLGQYLYGVYRTRLHENDWIHIDAGTGLLYLNQSLDHSSWEQLSIRNGGFPLLTVFLQVFLGSTAQREGECHWPGCARVYFSFINDTFPNCSSFKARDLCTPETGVSFRIRENRPPGTFYQFRMLPVQFLCPNISVKYKLLEGDGLPFRCDPDCLEVSTRWALDRELQEKYVLEAECAVAGPGANKEKVAVSFPVTVYDDEDDSPPTFSGGVGTASAVVEFKRKEGTVVATLQVFDADVVPASGELVRRYTSTLLSGDSWAQQTFRVEHTPNETLVQSNNNSVRATMHNYRLVLNRSLSISESRVLQLVVLVNDSDFQGPGSGFLFLHFNVSVLPVTLNLPMAYSFPVNRRARRYAQIGKVCVENCQEFSGVSIQYKLQPSSTNCSALGVVTSTEDTSGTLYVNDTEALRRPECTELQYTVVATDRQTRRQTQASLVVTVEGTYIAEEVGCPKSCAVNKRRPECEECGGLGSPTGRCEWRQGDGKGTTRNFSTCSPSTRTCPDGHCDALESRDINICPQDCLRGPIVGGHERGERQGIKAGYGICNCFPDEKKCFCEPEDSQAPLCDELCRTVITAAVLFSFIISVLLSTFCIHRYHKHXXKPPIXSAEMTFCRPAQGFPISYSSSGXRRPSLDSMENQVSVDSFKIPEDPKWEFPRKNLVLGKTLGEGEFGKVVKATAFRLKDRAGYTTVAVKMLKENASQSELRDLLSEFNLLKQVNHPHVIKLYGACSQDGPLLLIVEYAKYGSLRGFLRDSRKIGPAYVSSGGSRNSSSLDHPDERVLTMGDLISFAWQISRGMQYLAEMKLVHRDLAARNILVAEGRKMKISDFGLSRDVYEEDSYVKKSXGRIPVNWMAINSLSDHFYTTQSDVWSFGVLLWEIVTLGGNPYPGIPPERLFNLLKTGHRMERPNNCSEEMYRLMLQCWKQEPDKRPVFADISKDLEKMMVKSRDYLDLAASTPSDSLLYDDGLSEEETPLVDCNSAPLPRSLPSTWIENKLYGRISHAFTRF

SEQ ID NO: 13 - DNA, 3387 bp, Rattus norvegicus
  CDS (1)..(3384)
atggcgaaagcgaggtccggcgccgcagggctggggctgaagctgtttttgctgctgccgctactgggagaagccccgctgggtctctacttctcaagggatgcttactgggagaggctgtatgtggaccagccagctggcacacctctgctctatgtccatgccctacgggatgcccctggagaagtgcccagcttccgcctgggccagtatctctatggcgtctaccgcacgcgtctgcatgagaatgactggatccacatcgatgcaggcactggcctcctctacctcaatcagagcctggaccatagttcctgggagcagctcagcatccgaaatggcggcttccccttgctcaccgtcttcctccaggtcttcctggggtccacagcccagagagagggagagtgtcattggccaggctgtgcccgtgtgtacttctccttcatcaacgacaccttcccaaattgtagctccttcaaagcccgggatctctgcaccccggagacgggtgtgtccttccgcatcagggagaacaggccccctggcaccttctaccagttccgcatgctacctgtgcagttcctttgtcctaacatcagtgtgaagtacaaactcttagaaggggacggtctgcccttccgttgtgaccccgactgtctggaggtgagcacgcggtgggcgctggatcgggagcttcaggagaagtatgtgctggaggctgagtgcgcagtggcaggccctggagccaacaaggagaaggtggccgtgtccttcccggtgacggtgtatgatgaagacgactccccgcccaccttctccggaggtgtgggcaccgccagtgctgtggtggagtttaagcggaaggagggcactgtggtagccactctgcaggtgtttgatgcagatgtggtgccagcatctggggagctggtgaggcggtacacaagcacactactctcaggggattcctgggcccagcagaccttccgggtggagcacacacccaacgagaccttggtccagtccaacaacaactccgtgcgggcaaccatgcacaattacaagctggttctcaacaggagcctgtccatctcagagagccgagtcctgcagctagtagtcctggtcaatgactcagacttccaggggcctgggtcaggtgttctcttcctccatttcaacgtgtctgtgctgcctgtcaccctgaacctacccatggcctactccttcccagtgaataggagagcccgccgttatgcccagattgggaaagtttgcgtggagaactgccaggagttcagcggtgtctccatccagtacaagctgcagccctccagcaccaactgcagtgccctaggtgtggtcacctcaacagaagacacctcagggaccctatatgtaaatgacacggaggccctgcggcgacctgagtgtaccgagcttcagtacacagtggtagccactgaccggcagacccgcaggcagacccaggcttcgttagtcgtcacagtggaggggacatacattgcagaagaagtgggctgccccaagtcctgtgcagtaaacaagaggcgacctgagtgtgaggagtgtggtggcctgggttctccaactggcagatgtgagtggcgtcagggagatggtaaagggatcaccaggaacttctccacctgttctcctagcaccaggacctgtcctgatggccactgtgatgctctggagagccgggatatcaacatttgcccccaggactgtctccgtggccccattgttggcgggcatgagcgaggggagcgccaggggattaaagccggctatggcatctgcaactgtttccctgatgagaagaagtgcttctgcgagccagaggacagccagggcccattgtgcgatgagctgtgccgtacggtcatcacagccgctgtcctcttctccttcataatctctgtcctgctgtccaccttctgcatccaccgctaccacaagcatgcgcacaagccacccatcgcgtcagccgaaatgaccttctgccggccggcccagggcttcccaatcagctattcttcctcgggcacccgccggccctcactggactccatggagaaccaggtctctgtggactctttcaagatcccggaggatccgaagtgggaatttcctcggaagaacttagttcttgggaaaaccctgggagaaggcgagtttggaaaagtagtcaaggccacagccttccgtctgaaaggccgggcaggatacaccacagtggctgtgaaaatgctgaaagaaaacgcctcccagagtgaactacgagacctgctctctgagttcaaccttctgaaacaagtcaaccatccacatgtcatcaagttgtacggggcttgcagccaggatgggccacttcttctcattgtggagtatgcaaagtatggatccctgcgggggttcctgcgggacagccgcaagatcgggcctgcctatgtgagcagtggaggcagccgcaattccagctccctggaccacccagacgaaagggtgctgaccatgggcgacctcatctccttcgcctggcagatctcgaggggtatgcagtacttggctgaaatgaagctcgtacatcgagacttagctgccagaaacatcttggtggcagagggacggaagatgaagatctctgactttgggctgtcccgagatgtttatgaagaagattcctatgtgaagaaaagcaagggccggattcccgtcaaatggatggcaatcgagtctctcttcgatcacatctataccactcaaagtgatgtgtggtcctttggagtgctgctatgggagattgtgaccctgggaggcaacccctaccctggaattcctcctgaacgactcttcaaccttctgaagacaggccacaggatggagaggccagacaactgcagcgaggaaatgtaccgcctgatgctgcagtgctggaagcaggagccggacaagaggccagtatttgctgacatcagcaaggatctggagaagatgatggtcaaaagcagagactacttggacctggctgcatccaccccttcggactcactgctctatgacgatgggctctcggaagaggagacgcccctggtggactgtaacagtgctcccctcccgcgctccctcccttccacatggattgaaaacaaactctatggcatgtcagacccgaactggcctggagagagtcctgtaccactcacgagagccgatggcactagcactgggttcccaagatatgcaaatgatagtgtatatgctaactggatggtttcaccctcagcggcaaaattaatggacacatttgatagcgcggccgcagactacccatacgacgtcccagactacgcttag

SEQ ID NO: 14 - protein, 1128 aa, Rattus norvegicus
MAKARSGAAGLGLKLFLLLPLLGEAPLGLYFSRDAYWERLYVDQPAGTPLLYVHALRDAPGEVPSFRLGQYLYGVYRTRLHENDWIHIDAGTGLLYLNQSLDHSSWEQLSIRNGGFPLLTVFLQVFLGSTAQREGECHWPGCARVYFSFINDTFPNCSSFKARDLCTPETGVSFRIRENRPPGTFYQFRMLPVQFLCPNISVKYKLLEGDGLPFRCDPDCLEVSTRWALDRELQEKYVLEAECAVAGPGANKEKVAVSFPVTVYDEDDSPPTFSGGVGTASAVVEFKRKEGTVVATLQVFDADVVPASGELVRRYTSTLLSGDSWAQQTFRVEHTPNETLVQSNNNSVRATMHNYKLVLNRSLSISESRVLQLVVLVNDSDFQGPGSGVLFLHFNVSVLPVTLNLPMAYSFPVNRRARRYAQIGKVCVENCQEFSGVSIQYKLQPSSTNCSALGVVTSTEDTSGTLYVNDTEALRRPECTELQYTVVATDRQTRRQTQASLVVTVEGTYIAEEVGCPKSCAVNKRRPECEECGGLGSPTGRCEWRQGDGKGITRNFSTCSPSTRTCPDGHCDALESRDINICPQDCLRGPIVGGHERGERQGIKAGYGICNCFPDEKKCFCEPEDSQGPLCDELCRTVITAAVLFSFIISVLLSTFCIHRYHKHAHKPPIASAEMTFCRPAQGFPISYSSSGTRRPSLDSMENQVSVDSFKIPEDPKWEFPRKNLVLGKTLGEGEFGKVVKATAFRLKGRAGYTTVAVKMLKENASQSELRDLLSEFNLLKQVNHPHVIKLYGACSQDGPLLLIVEYAKYGSLRGFLRDSRKIGPAYVSSGGSRNSSSLDHPDERVLTMGDLISFAWQISRGMQYLAEMKLVHRDLAARNILVAEGRKMKISDFGLSRDVYEEDSYVKKSKGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKSRDYLDLAASTPSDSLLYDDGLSEEETPLVDCNSAPLPRSLPSTWIENKLYGMSDPNWPGESPVPLTRADGTSTGFPRYANDSVYANWMVSPSAAKLMDTFDSAAADYPYDVPDYA

SEQ ID NO: 15 - DNA, 3348 bp, Mus musculus
  CDS (1)..(3345)
atggcgaaagcgacgtccggcgccgcagggctggggctgaagctgattttgctcctgccgctgctaggagaagccccactgggcctctatttctcaagggatgcttactgggagaggctgtatgtagaccagccagctggcacacctctgctctatgtccatgccctacgggatgcccctggagaagtgccgagcttccgcctgggccagcatctctatggcgtctaccgtacacggctgcatgagaatgactggatccgcatcaatgagactactggccttctctacctcaatcagagcctggaccacagttcctgggaacagctcagcatccgcaatggtggtttccccctgctcaccatcttcctccaggtctttctggggtccacagcccagagagagggagaatgccattggccaggctgtacccgtgtgtacttctccttcatcaacgacaccttcccaaattgtagctccttcaaagcccaggatctctgcatcccagagacagccgtgtccttccgagtcagggagaacaggcctcctggcaccttctaccacttccacatgttacccgtgcagttcctttgtcccaacatcagtgtgaagtacagtctcttaggaggggatagtctgcccttccgttgtgacccagactgcctggaggtgagcactcgctgggccctggatcgagagctccgggagaagtatgtgctggaggctttgtgcatagtggcaggccctggtgccaacaaagagacggtgactctgtccttcccagtgacagtgtatgatgaggacgactcggcgcccaccttctctggaggtgtgggcactgccagcgcggtggtggagtttaagcggaaggagggcactgtggtggccaccctgcaggtgttcgatgcagatgtggtgccagcgtctggggagctggtgagacggtacacaaacacactcctctcaggggactcctgggcccagcagaccttccgggtggagcattcgcccatcgagaccttggtccaggtcaacaacaactccgttcgggcaaccatgcacaattacaagctgattctcaacaggagcctgtctatctcagagagccgagtcctgcagctcgcggtcctggtcaacgactcagacttccaggggcctggggcaggtggtatcctcgtcctccatttcaacgtgtctgtactgcccgtcaccctgaacctacccagggcctactccttcccagtgaataagagggcccgccgctatgcccagatcgggaaagtctgtgtggaaaactgccaggagttcagcggtgtctccatccagtacaagctgcagccttccagcatcaactgcactgccctaggtgtggtcacctcacccgaggacacctcggggaccctatttgtaaatgacacagaggccctgcggcgacctgagtgcaccaagcttcagtacacggtggtagccactgaccggcagacccgcagacagacccaggcttcgctagtggtcactgtggaggggacatccattactgaagaagtaggctgccccaagtcctgtgcagtaaacaagaggcgccccgagtgtgaggaatgtggtggcctgggttctccaactggcaggtgcgagtggcgccagggagatggtaaagggatcaccaggaacttctccacctgctcccccagtaccaggacctgccccgacggccactgtgatgctgtggagagccgggatgccaacatttgcccccaggactgtctccgtgccgacattgttggaggacacgagcgaggggagcgccagggcattaaagcaggctacggcatctgcaactgtttccctgatgagaagaaatgcttctgcgagccagaggacagccagggcccactgtgtgatgcgctgtgccgcacgatcatcacagctgccctcttctcccttatcatctccatcctgctgtccatcttctgtgtctgccaccaccacaagcatgggcacaagccgcccattgcatcagcggaaatgaccttctgccggccggcccagggcttcccaatcagttattcctcctcaggcacccgccggccctcactggattccacggagaaccaggttcctgtggactctttcaagatcccggaggatccaaagtgggaatttcctcggaagaacttagttcttgggaaaactctgggagaaggcgagtttggaaaagttgtcaaggccacagccttccgtctgaaaggccgggcaggatacaccacagtggctgtgaaaatgctgaaagaaaacgcctcccagagtgagttacgagacctgctgtctgagttcaaccttctgaaacaagtcaaccatccacatgtcatcaagttgtatggggcctgcagccaggatgggccacttcttctcattgtggagtatgccaagtatggctccctgcggggattcctccgtgacagccgcaagattgggcctgcctatgtgagcggtggaggcagccgcaactccagctcgctggaccacccagatgaaagggtactgaccatgggtgacctcatctccttcgcctggcagatctcgaggggtatgcagtacttggcagaaatgaagcttgtacatcgggacttagctgccaggaacatcttggtggctgagggacggaagatgaagatttccgactttgggctgtcccgagatgtttatgaggaagattcctatgtgaagaaaagcaagggccggattcccgtcaagtggatggcaattgagtcccttttcgatcacatctatactactcaaagtgatgtgtggtcctttggagtgctgctctgggagattgtgaccctgggaggcaacccctaccctggaattcctcctgaacgactcttcaaccttctgaagacaggccacaggatggagaggccagacaactgcagcgaggaaatgtaccgtctgatgctgcagtgctggaagcaggagccagacaagaggccagtgtttgctgacatcagcaaggatctggagaagatgatggtcaagagcagagactacttggacctggctgcatccacaccttcggactcactgctgtatgacgatgggctctcagaagaggagacacccctggtggactgtaacaatgctcccctcccgcgctccctcccttccacatggattgaaaacaaactctatggcatgtcagacccgaactggcctggagagagtcctgtaccactcacgagagccgatggcactagcactgggttcccaagatatgcaaatgatagtgtatatgctaactggatggtttcaccctcagcggcaaaattaatggacacatttgatagctaa

SEQ ID NO: 16 - protein, 1115 aa, Mus musculus
MAKATSGAAGLGLKLILLLPLLGEAPLGLYFSRDAYWERLYVDQPAGTPLLYVHALRDAPGEVPSFRLGQHLYGVYRTRLHENDWIRINETTGLLYLNQSLDHSSWEQLSIRNGGFPLLTIFLQVFLGSTAQREGECHWPGCTRVYFSFINDTFPNCSSFKAQDLCIPETAVSFRVRENRPPGTFYHFHMLPVQFLCPNISVKYSLLGGDSLPFRCDPDCLEVSTRWALDRELREKYVLEALCIVAGPGANKETVTLSFPVTVYDEDDSAPTFSGGVGTASAVVEFKRKEGTVVATLQVFDADVVPASGELVRRYTNTLLSGDSWAQQTFRVEHSPIETLVQVNNNSVRATMHNYKLILNRSLSISESRVLQLAVLVNDSDFQGPGAGGILVLHFNVSVLPVTLNLPRAYSFPVNKRARRYAQIGKVCVENCQEFSGVSIQYKLQPSSINCTALGVVTSPEDTSGTLFVNDTEALRRPECTKLQYTVVATDRQTRRQTQASLVVTVEGTSITEEVGCPKSCAVNKRRPECEECGGLGSPTGRCEWRQGDGKGITRNFSTCSPSTRTCPDGHCDAVESRDANICPQDCLRADIVGGHERGERQGIKAGYGICNCFPDEKKCFCEPEDSQGPLCDALCRTIITAALFSLIISILLSIFCVCHHHKHGHKPPIASAEMTFCRPAQGFPISYSSSGTRRPSLDSTENQVPVDSFKIPEDPKWEFPRKNLVLGKTLGEGEFGKVVKATAFRLKGRAGYTTVAVKMLKENASQSELRDLLSEFNLLKQVNHPHVIKLYGACSQDGPLLLIVEYAKYGSLRGFLRDSRKIGPAYVSGGGSRNSSSLDHPDERVLTMGDLISFAWQISRGMQYLAEMKLVHRDLAARNILVAEGRKMKISDFGLSRDVYEEDSYVKKSKGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKSRDYLDLAASTPSDSLLYDDGLSEEETPLVDCNNAPLPRSLPSTWIENKLYGMSDPNWPGESPVPLTRADGTSTGFPRYANDSVYANWMVSPSAAKLMDTFDS

SEQ ID NO: 17 - DNA, 3348 bp, Mus musculus
  CDS (1)..(3345)
atggcgaaagcgacgtccggcgccgcagggctggggctgaagctgattttgctcctgccgctgctaggagaagccccactgggcctctatttctcaagggatgcttactgggagaggctgtatgtagaccagccagctggcacacctctgctctatgtccatgccctacgggatgcccctggagaagtgccgagcttccgcctgggccagcatctctatggcgtctaccgtacacggctgcatgagaatgactggatccgcatcaatgagactactggccttctctacctcaatcagagcctggaccacagttcctgggaacagctcagcatccgcaatggtggtttccccctgctcaccatcttcctccaggtctttctggggtccacagcccagagagagggagaatgccattggccaggctgtacccgtgtgtacttctccttcatcaacgacaccttcccaaattgtagctccttcaaagcccaggatctctgcatcccagagacagccgtgtcctcccgagtcagggagaacagscctcctggcaccttctaccacttccacatgttacccgtgcagttcctttgtcccaacatcagtgtgaagtacagtctcttaggaggggatagtctgcccttccgttgtgacccagactgcctggaggtgagcactcgctgggccctggatcgagagctccgggagaagtatgtgctggaggctttgtgcatagtggcaggccctggtgccaacaaagagacggtgactctgtccttcccagtgacagtgtatgatgaggacgactcggcgcccaccttctctggaggtgtgggcactgccagcgcggtggtggagtttaagcggaaggagggcactgtggtggccaccctgcaggtgttcgatgcagatgtggtgccagcgtctggggagctggtgagacggtacacaaacacactcctctcaggggactcctgggcccagcagaccttccgggtggagcattcgcccatcgagaccttggtccaggtcaacaacaactccgttcgggcaaccatgcacaattacaagctgattctcaacaggagcctgtctatctcagagagccgagtcctgcagctcgcggtcctggtcaacgactcagacttccaggggcctggggcaggtggtatcctcgtcctccatttcaacgtgtctgtactgcccgtcaccctgaacctacccagggcctactccttcccagtgaataagagggcccgccgctatgcccagatcgggaaagtctgtgtggaaaactgccaggagttcagcggtgtctccatccagtacaagctgcagccttccagcatcaactgcactgccctaggtgtggtcacctcacccgaggacacctcggggaccctatttgtaaatgacacagaggccctgcggcgacctgagtgcaccaagcttcagtacacggtggtagccactgaccggcagacccgcagacagacccaggcttcgctagtggtcactgtggaggggacatccattactgaagaagtaggctgccccaagtcctgtgcagtaaacaagaggcgccccgagtgtgaggaatgtggtggcctgggttctccaactggcaggtgcgagtggcgccagggagatggtaaagggatcaccaggaacttctccacctgctcccccagtaccaggacctgccccgacggccactgtgatgctgtggagagccgggatgccaacatttgcccccaggactgtctccgtgccgacattgttggaggacacgagcgaggggagcgccagggcattaaagcaggctacggcatctgcaactgtttccctgatgagaagaaatgcttctgcgagccagaggacagccagggcccactgtgtgatgcgctgtgccgcacgatcatcacagctgccctcttctcccttatcatctccatcctgctgtccatcttctgtgtctgccaccaccacaagcatgggcacaagccgcccattgcatcagcggaaatgaccttctgccggccggcccagggcttcccaatcagttattcctcctcaggcacccgccggccctcactggattccacggagaaccaggttcctgtggactctttcaagatcccggaggatccaaagtgggaatttcctcggaagaacttagttcttgggaaaactctgggagaaggcgagtttggaaaagttgtcvaggccacagccttccgtctgaaaggccgggcaggatacaccacagtggctgtgaaaatsctgaaagaaaacgcctcccagagtgagttacgagacctgctgtctgagttcaaccttctgaaacaagtcaaccatccacatgtcatcaagttgtatggggcctgcagccaggatgggccacttcttctcattgtggagtatgccaagtatggctctctgcggggattcctccgtgacagccgcaagattgggcctgcctatgtgagcggtggaggcagccgcaactccagctcgctggaccacccagatgaaagggtactgaccatgggtgacctcatctccttcgcctggcagatctcgaggggtatgcagtacttggcagaaatgaagcttgtacatcgggacttagctgccaggaacatcttggtggctgagggacggaagatgaagatttccgactttgggctgtcccgagatgtttatgaggaagattcctatgtgaagaaaagcaagggccggattcccgtcaagtggatggcaattgagtcccttttcgatcacatctatactactcaaagtgatgtgtggtcctttggagtgctgctctgggagattgtgaccctgggaggcaacccctaccctggaattcctcctgaacgactcttcaaccttctgaagacaggccacaggatggagaggccagacaactgcagcgaggaaatgtaccgtctgatgctgcagtgctggaagcaggagccagacaagaggccagtgtttgctgacatcagcaaggatctggagaagatgatggtcaagagcagagactacttggacctggctgcatccacaccttcggactcactgctgtatgacgatgggctctcagaagaggagacacccctggtggactgtaacaatgctcccctcccgcgctccctcccttccacatggattgaaaacaaactctatggcatgtcagacccgaactggcctggagagagtcctgtaccactcacgagagccgatggcactagcactgggttcccaagatatgcaaatgatagtgtatatgctaactggatggtttcaccctcagcggcaaaattaatggacacatttgatagctaa

SEQ ID NO: 18 - protein, 1115 aa, Mus musculus
  misc_feature (180)..(180): The ′Xaa′ at location 180 stands for Arg, or Ser.
MAKATSGAAGLGLKLILLLPLLGEAPLGLYFSRDAYWERLYVDQPAGTPLLYVHALRDAPGEVPSFRLGQHLYGVYRTRLHENDWIRINETTGLLYLNQSLDHSSWEQLSIRNGGFPLLTIFLQVFLGSTAQREGECHWPGCTRVYFSFINDTFPNCSSFKAQDLCIPETAVSSRVRENXPPGTFYHFHMLPVQFLCPNISVKYSLLGGDSLPFRCDPDCLEVSTRWALDRELREKYVLEALCIVAGPGANKETVTLSFPVTVYDEDDSAPTFSGGVGTASAVVEFKRKEGTVVATLQVFDADVVPASGELVRRYTNTLLSGDSWAQQTFRVEHSPIETLVQVNNNSVRATMHNYKLILNRSLSISESRVLQLAVLVNDSDFQGPGAGGILVLHFNVSVLPVTLNLPRAYSFPVNKRARRYAQIGKVCVENCQEFSGVSIQYKLQPSSINCTALGVVTSPEDTSGTLFVNDTEALRRPECTKLQYTVVATDRQTRRQTQASLVVTVEGTSITEEVGCPKSCAVNKRRPECEECGGLGSPTGRCEWRQGDGKGITRNFSTCSPSTRTCPDGHCDAVESRDANICPQDCLRADIVGGHERGERQGIKAGYGICNCFPDEKKCFCEPEDSQGPLCDALCRTIITAALFSLIISILLSIFCVCHHHKHGHKPPIASAEMTFCRPAQGFPISYSSSGTRRPSLDSTENQVPVDSFKIPEDPKWEFPRKNLVLGKTLGEGEFGKVVXATAFRLKGRAGYTTVAVKXLKENASQSELRDLLSEFNLLKQVNHPHVIKLYGACSQDGPLLLIVEYAKYGSLRGFLRDSRKIGPAYVSGGGSRNSSSLDHPDERVLTMGDLISFAWQISRGMQYLAEMKLVHRDLAARNILVAEGRKMKISDFGLSRDVYEEDSYVKKSKGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKSRDYLDLAASTPSDSLLYDDGLSEEETPLVDCNNAPLPRSLPSTWIENKLYGMSDPNWPGESPVPLTRADGTSTGFPRYANDSVYANWMVSPSAAKLMDTFDS

SEQ ID NO: 19 - DNA, 3222 bp, Mus musculus
  CDS (1)..(3219)
atggcgaaagcgacgtccggcgccgcagggctggggctgaagctgattttgctcctgccgctgctaggagaagccccactgggcctctatttctcaagggatgcttactgggagaggctgtatgtagaccagccagctggcacacctctgctctatgtccatgccctacgggatgcccctggagaagtgccgagcttccgcctgggccagcatctctatggcgtctaccgtacacggctgcatgagaatgactggatccgcatcaatgagactactggccttctctacctcaatcagagcctggaccacagttcctgggaacagctcagcatccgcaatggtggtttccccctgctcaccatcttcctccaggtctttctggggtccacagcccagagagagggagaatgccattggccaggctgtacccgtgtgtacttctccttcatcaacgacaccttcccaaattgtagctccttcaaagcccaggatctctgcatcccagagacagccgtgtccttccgagtcagggagaacaggcctcctggcaccttctaccacttccacatgttacccgtgcagttcctttgtcccaacatcagtgtgaagtacagtctcttaggaggggatagtctgcccttccgttgtgacccagactgcctggaggtgagcactcgctgggccctggatcgagagctccgggagaagtatgtgctggaggctttgtgcatagtggcaggccctggtgccaacaaagagacggtgactctgtccttcccagtgacagtgtatgatgaggacgactcggcgcccaccttctctggaggtgtgggcactgccagcgcggtggtggagtttaagcggaaggagggcactgtggtggccaccctgcaggtgttcgatgcagatgtggtgccagcgtctggggagctggtgagacggtacacaaacacactcctctcaggggactcctgggcccagcagaccttccgggtggagcattcgcccatcgagaccttggtccaggtcaacaacaactccgttcgggcaaccatgcacaattacaagctgattctcaacaggagcctgtctatctcagagagccgagtcctgcagctcgcggtcctggtcaacgactcagacttccaggggcctggggcaggtggtatcctcgtcctccatttcaacgtgtctgtactgcccgtcaccctgaacctacccagggcctactccttcccagtgaataagagggcccgccgctatgcccagatcgggaaagtctgtgtggaaaactgccaggagttcagcggtgtctccatccagtacaagctgcagccttccagcatcaactgcactgccctaggtgtggtcacctcacccgaggacacctcggggaccctatttgtaaatgacacagaggccctgcggcgacctgagtgcaccaagcttcagtacacggtggtagccactgaccggcagacccgcagacagacccaggcttcgctagtggtcactgtggaggggacatccattactgaagaagtaggctgccccaagtcctgtgcagtaaacaagaggcgccccgagtgtgaggaatgtggtggcctgggttctccaactggcaggtgcgagtggcgccagggagatggtaaagggatcaccaggaacttctccacctgctcccccagtaccaggacctgccccgacggccactgtgatgctgtggagagccgggatgccaacatttgcccccaggactgtctccgtgccgacattgttggaggacacgagcgaggggagcgccagggcattaaagcaggctacggcatctgcaactgtttccctgatgagaagaaatgcttctgcgagccagaggacagccagggcccactgtgtgatgcgctgtgccgcacgatcatcacagctgccctcttctcccttatcatctccatcctgctgtccatcttctgtgtctgccaccaccacaagcatgggcacaagccgcccattgcatcagcggaaatgaccttctgccggccggcccagggcttcccaatcagttattcctcctcaggcacccgccggccctcactggattccacggagaaccaggttcctgtggactctttcaagatcccggaggatccaaagtgggaatttcctcggaagaacttagttcttgggaaaactctgggagaaggcgagtttggaaaagttgtcaaggccacagccttccgtctgaaaggccgggcaggatacaccacagtggctgtgaaaatgctgaaagaaaacgcctcccagagtgagttacgagacctgctgtctgagttcaaccttctgaaacaagtcaaccatccacatgtcatcaagttgtatggggcctgcagccaggatgggccacttcttctcattgtggagtatgccaagtatggctccctgcggggattcctccgtgacagccgcaagattgggcctgcctatgtgagcggtggaggcagccgcaactccagctcgctggaccacccagatgaaagggtactgaccatgggtgacctcatctccttcgcctggcagatctcgaggggtatgcagtacttggcagaaatgaagcttgtacatcgggacttagctgccaggaacatcttggtggctgagggacggaagatgaagatttccgactttgggctgtcccgagatgtttatgaggaagattcctatgtgaagaaaagcaagggccggattcccgtcaagtggatggcaattgagtcccttttcgatcacatctatactactcaaagtgatgtgtggtcctttggagtgctgctctgggagattgtgaccctgggaggcaacccctaccctggaattcctcctgaacgactcttcaaccttctgaagacaggccacaggatggagaggccagacaactgcagcgaggaaatgtaccgtctgatgctgcagtgctggaagcaggagccagacaagaggccagtgtttgctgacatcagcaaggatctggagaagatgatggtcaagagcagagactacttggacctggctgcatccacaccttcggactcactgctgtatgacgatgggctctcagaagaggagacacccctggtggactgtaacaatgctcccctcccgcgctccctcccttccacatggattgaaaacaaactctatggtagaatttcacatgcatttactagattctag

SEQ ID NO: 20 - protein, 1073 aa, Mus musculus
MAKATSGAAGLGLKLILLLPLLGEAPLGLYFSRDAYWERLYVDQPAGTPLLYVHALRDAPGEVPSFRLGQHLYGVYRTRLHENDWIRINETTGLLYLNQSLDHSSWEQLSIRNGGFPLLTIFLQVFLGSTAQREGECHWPGCTRVYFSFINDTFPNCSSFKAQDLCIPETAVSFRVRENRPPGTFYHFHMLPVQFLCPNISVKYSLLGGDSLPFRCDPDCLEVSTRWALDRELREKYVLEALCIVAGPGANKETVTLSFPVTVYDEDDSAPTFSGGVGTASAVVEFKRKEGTVVATLQVFDADVVPASGELVRRYTNTLLSGDSWAQQTFRVEHSPIETLVQVNNNSVRATMHNYKLILNRSLSISESRVLQLAVLVNDSDFQGPGAGGILVLHFNVSVLPVTLNLPRAYSFPVNKRARRYAQIGKVCVENCQEFSGVSIQYKLQPSSINCTALGVVTSPEDTSGTLFVNDTEALRRPECTKLQYTVVATDRQTRRQTQASLVVTVEGTSITEEVGCPKSCAVNKRRPECEECGGLGSPTGRCEWRQGDGKGITRNFSTCSPSTRTCPDGHCDAVESRDANICPQDCLRADIVGGHERGERQGIKAGYGICNCFPDEKKCFCEPEDSQGPLCDALCRTIITAALFSLIISILLSIFCVCHHHKHGHKPPIASAEMTFCRPAQGFPISYSSSGTRRPSLDSTENQVPVDSFKIPEDPKWEFPRKNLVLGKTLGEGEFGKVVKATAFRLKGRAGYTTVAVKMLKENASQSELRDLLSEFNLLKQVNHPHVIKLYGACSQDGPLLLIVEYAKYGSLRGFLRDSRKIGPAYVSGGGSRNSSSLDHPDERVLTMGDLISFAWQISRGMQYLAEMKLVHRDLAARNILVAEGRKMKISDFGLSRDVYEEDSYVKKSKGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKSRDYLDLAASTPSDSLLYDDGLSEEETPLVDCNNAPLPRSLPSTWIENKLYGRISHAFTRF

SEQ ID NO: 21 - DNA, 2388 bp, Mus musculus
  CDS (1)..(2385)
atgtccatgccctacgggatgcccctggagaagtgccgagcttccgcctgggccagcatctctatggcgtctaccgtacacggctgcatgagaatgactggatccgcatcaatgagactactggccttctctacctcaatcagagcctggaccacagttcctgggaacagctcagcatccgcaatgggtggtttccccctgctcaccatcttcctccaggtctttctggggtccacagcccagagagagggagaatgccattggccaggctgtacccgtgtgtacttctccttcatcaacgacaccttcccaaattgtagctccttcaaagcccaggatctctgcatcccagagacagccgtgtccttccgagtcagggagaacaggcctcctggcaccttctaccacttccacatgttacccgtgcagttcctttgtcccaacatcagtgtgaagtacagtctcttaggaggggatagtctgcccttccgttgtgacccagactgcctggaggtgagcactcgctgggccctggatcgagagctccgggagaagtatgtgctggaggctttgtgcatagtggcaggccctggtgccaacaaagagacggtgactctgtccttcccggtgacagtgtatgatgaggacgactcggcgcccaccttctctggaggtgtgggcactgccagcgcggtggtggagtttaagcggaaggagggcactgtggtggccaccctgcaggtgttcgatgcagatgtggtgccagcgtctggggagctggtgagacggtacacaaacacactcctctcaggggactcctgggcccagcagaccttccgggtggagcattcgcccatcgagaccttggtccaggtcaacaacaactccgttcgggcaaccatgcacaattacaagctgattctcaacaggagcctgtctatctcagagagccgagtcctgcagctcgcggtcctggtcaacgactcagacttccaggggcctggggcaggtggtatcctcgtcctccatttcaacgtgtctgtactgcccgtcaccctgaacctacccagggcctactccttcccagtgaataagagggcccgccgctatgcccagatcgggaaagtctgtgtggaaaactgccaggagttcagcggtgtctccatccagtacaagctgcagccttccagcatcaactgcactgccctaggtgtggtcacctcacccgaggacacctcggggaccctatttgtaaatgacacagaggccctgcggcgacctgagtgcaccaagcttcagtacacggtggtagccactgaccggcagacccgcagacagacccaggcttcgctagtggtcactgtggaggggacatccattactgaagaagtaggctgccccaagtcctgtgcagtaaacaagaggcgccccgagtgtgaggaatgtggtggcctgggttctccaactggcaggtgcgagtggcgccagggagatggtaaagggatcaccaggaacttctccacctgctcccccagtaccaggacctgccccgacggccactgtgatgctgtggagagccgggatgccaacatttgcccccaggactgtctccgtgccgacattgttggaggacacgagcgaggggagcgccagggcattaaagcaggctacggcatctgcaactgtttccctgatgagaagaaatgcttctgcgagccagaggacagccagggcccactgtgtgatgcgctgtgccgcacgatcatcacagctgccctcttctcccttatcatctccatcctgctgtccatcttctgtgtctgccaccaccacaagcatgggcacaagccgcccattgcatcagcggaaatgaccttctgccggccggcccagggcttcccaatcagttattcctcctcaggcacccgccggccctcactggattccacggagaaccaggttcctgtggactctttcaagatcccggaggatccaaagtgggaatttcctcggaagaacttagttcttgggaaaactctgggagaaggcgagtttggaaaagttgtcaaggccacagccttccgtctgaaaggccgggcaggatacaccacagtggctgtgaaaatgctgaaagaaaacgcctcccagagtgagttacgagacctgctgtctgagttcaaccttctgaaacaagtcaaccatccacatgtcatcaagttgtatggggcctgcagccaggatgggccacttcttctcattgtggagtatgccaagtatggctccctgcggggattcctccgtgacagccgcaagattgggcctgcctatgtgagcggtggaggcagccgcaactccagctcgctggaccacccagatgaagggtactga

SEQ ID NO: 22 - protein, 795 aa, Mus musculus
MSMPYGMPLEKCRASAWASISMASTVHGCMRMTGSASMRLLAFSTSIRAWTTVPGNSSASAMGGFPLLTIFLQVFLGSTAQREGECHWPGCTRVYFSFINDTFPNCSSFKAQDLCIPETAVSFRVRENRPPGTFYHFHMLPVQFLCPNISVKYSLLGGDSLPFRCDPDCLEVSTRWALDRELREKYVLEALCIVAGPGANKETVTLSFPVTVYDEDDSAPTFSGGVGTASAVVEFKRKEGTVVATLQVFDADVVPASGELVRRYTNTLLSGDSWAQQTFRVEHSPIETLVQVNNNSVRATMHNYKLILNRSLSISESRVLQLAVLVNDSDFQGPGAGGILVLHFNVSVLPVTLNLPRAYSFPVNKRARRYAQIGKVCVENCQEFSGVSIQYKLQPSSINCTALGVVTSPEDTSGTLFVNDTEALRRPECTKLQYTVVATDRQTRRQTQASLVVTVEGTSITEEVGCPKSCAVNKRRPECEECGGLGSPTGRCEWRQGDGKGITRNFSTCSPSTRTCPDGHCDAVESRDANICPQDCLRADIVGGHERGERQGIKAGYGICNCFPDEKKCFCEPEDSQGPLCDALCRTIITAALFSLIISILLSIFCVCHHHKHGHKPPIASAEMTFCRPAQGFPISYSSSGTRRPSLDSTENQVPVDSFKIPEDPKWEFPRKNLVLGKTLGEGEFGKVVKATAFRLKGRAGYTTVAVKMLKENASQSELRDLLSEFNLLKQVNHPHVIKLYGACSQDGPLLLIVEYAKYGSLRGFLRDSRKIGPAYVSGGGSRNSSSLDHPDEGY

SEQ ID NO: 23 - DNA, 3327 bp, Canis lupus familiaris
  CDS (1)..(3324)
atggcagcatctcggatcattaagggttggttcgtgcccttctgccaggctccactgggactctacttctccagggatgcttattgggagaagctgtatgtggaccagccatctggcatgcccctgctctatgtccatgccctgcgggacatccccgaggaggtgcctagcttccgcctaggccagcacctttacggcatcgcctaccgtgcaaggctgcatgagaacgactggatccgcatcgaggaggacacaggccttctctaccttaaccggagcctggatcacagcacctgggagaagctcagcatccgcaatggcggcttccctgtgctcaccatctacgtccaggtcttcctgtcatctgcatccctgcgtgagggcgagtgccagtggccaggctgtgcccgtgtgtacttctccttcattaacacctccttcccagcttgtggttccctcaaaccccgggagctctgcttccctgagactggtgtctccttccgcattcgagagaacaggcctcccggcaccttccaccaattccgtctgctgcccatgcagttcctctgccccaacatcagcgtgtcctacaagctcctagaaggtgagaatctgcccttccgttgcgccccggacagcctggaggtgagcacacgctgggccctggaccgtgagctgcgggagaagtatgagctggtggccgcgtgcaccgtgcgcgtcggtgcgcgtaaagaggaggtggtgatggtgcccttccccgtgaccgtgtatgacgaggacgactcggcgcccaccttcctcgggggcttcgacactgccagcgccgtggtggagttcaagaggaaggagggcactgtggtggccacaatacatgtctttgacgcggacgtggtaccagcatctggggagctcctgaggcgatacacaagtacactactccctggggatgcctgggccctccagacatttcgtgtggaacactcacccaacgagaccttggtccaggccaacggcagctttgtgcgggcaactgtgcatgactacaggctggttctcaaccggagcctccccatctcggagagccgctccctgcagctggccgttctggtcaatgactcggacttccagggtcccggggagggcatcctcttgctccacttcaacgtgacggtgctgcctgtcagcctgcacttacccagcgcctacaccttcactgtgagcagacgggcccgccgctttgcccagattgggaaagtctgtgtggaaaactgccaggagttcagtggcatccacgtgcagtacaagctgcagctctccagcaccaactgtagcgtcctgggggtggtcacgtcagccgaggacaccacagggaccctgtacgtgaatgacacggaagccctgcagcggctcgattgttctcaactccagtacacagtggtggctgccgaccggccaacccgcaggcagacccaggccccgctggtcgtcaccgtggaggggacatatgtggctgaggagccaggctgccccctgtcctgtgcagtcagcaagagacggcctgagtgtgaagagtgcggcggcctgggctctctgacgggcaagtgcgagtggagacagggagatggcaaagggatcaccagaaacttctccacctgctcccccaatgtcaagacctgccctgatggccactgtgatgctgtagagagcaggaatgtcaacatctgcccccaggactgcctccgtggcagcatcatcggtgggcatgagccaggggaccgctgggggataaaagctggcttcggaatctgcaactgttttcctgaagagaagaagtgcttctgcgagcctgaagacagccaggacccactgtgtgatgagctctgccgcacggtgattgcagcggctgtgctcttctccttcatcgtctccatgctgctctccaccttctgcatccaccgctaccacaagaatgcccacaagccgcccatcgcctctgccgaaatgaccttccgccggcccgcccaggccttcccagtcagctactcctcatcaggcgcccgccggccctcactggactccatggagaaccaggtctctgtggatgccttcaaaatcccggaggatcctaagtgggaattccctcggaagaacttagttcttggaaaaactctgggagaaggtgaatttggaaaagtggttaaggcaacagctttccggctaaaaggcaaagcaggatacacgaccgttgccgtgaagatgctgaaagagaatgcttccccaagcgagctgcgggatctgctatcagaattcaatctcctgaagcaggtcaaccacccgcatgtcatcaagctgtacggagcctgcagccaggatgggccactcttcctcattgtggagtatgccaagtatggctcactgcggggcttcctccgagaaagccgcaaggcgggtccaggctacgtgggcagtggaggcagccgaagctccagctacctggacaaccccgaggagcgggcgctgaccatgggcgacctcatctccttcgcctggcagatctctcgggggatgcggtacctggcagagatgaagcttgtccatcgggacttggccgccagaaacgtcctggtggctgaggggcggaagatgaagatttcagattttggcctctcccgagatgtttatgaggaggattcctacgtgaagaggagcaagggtcggattccagtcaagtggatggcaattgaatctcttttcgatcatatctataccacccaaagtgatgtgtggtccttcggtgtcctgctgtgggagattgtgactctggggggcaacccctaccctgggattcctccagagcggctcttcaaccttctgaagacaggctaccggatggagaggcctgacaactgcagcgaggagatgtatggtctaatgctgcagtgttggaagcaggaaccagacaagaggccagtgtttgctgacatcagcaaagacctagagaagatgatggttaagaacagagactacttggacctggcggcgtccaccccatctgactccctgctttatgacgatggcctctcggaggaggagacacctctggtggactgtaataatgctcccctccctcgaacccttccctccacgtggattgaaaacaaactctatggcatgtcagacccgaactggcctgaagagagtcctgtaccactcacgagagctgatggcactaactctgtgtgtccaagatatgcaaatgatagtgtatatgctaactggatggtttcaccctcagcggcacaattaatggacgcatttgatagttaa

SEQ ID NO: 24 - protein, 1108 aa, Canis lupus familiaris
MAASRIIKGWFVPFCQAPLGLYFSRDAYWEKLYVDQPSGMPLLYVHALRDIPEEVPSFRLGQHLYGIAYRARLHENDWIRIEEDTGLLYLNRSLDHSTWEKLSIRNGGFPVLTIYVQVFLSSASLREGECQWPGCARVYFSFINTSFPACGSLKPRELCFPETGVSFRIRENRPPGTFHQFRLLPMQFLCPNISVSYKLLEGENLPFRCAPDSLEVSTRWALDRELREKYELVAACTVRVGARKEEVVMVPFPVTVYDEDDSAPTFLGGFDTASAVVEFKRKEGTVVATIHVFDADVVPASGELLRRYTSTLLPGDAWALQTFRVEHSPNETLVQANGSFVRATVHDYRLVLNRSLPISESRSLQLAVLVNDSDFQGPGEGILLLHFNVTVLPVSLHLPSAYTFTVSRRARRFAQIGKVCVENCQEFSGIHVQYKLQLSSTNCSVLGVVTSAEDTTGTLYVNDTEALQRLDCSQLQYTVVAADRPTRRQTQAPLVVTVEGTYVAEEPGCPLSCAVSKRRPECEECGGLGSLTGKCEWRQGDGKGITRNFSTCSPNVKTCPDGHCDAVESRNVNICPQDCLRGSIIGGHEPGDRWGIKAGFGICNCFPEEKKCFCEPEDSQDPLCDELCRTVIAAAVLFSFIVSMLLSTFCIHRYHKNAHKPPIASAEMTFRRPAQAFPVSYSSSGARRPSLDSMENQVSVDAFKIPEDPKWEFPRKNLVLGKTLGEGEFGKVVKATAFRLKGKAGYTTVAVKMLKENASPSELRDLLSEFNLLKQVNHPHVIKLYGACSQDGPLFLIVEYAKYGSLRGFLRESRKAGPGYVGSGGSRSSSYLDNPEERALTMGDLISFAWQISRGMRYLAEMKLVHRDLAARNVLVAEGRKMKISDFGLSRDVYEEDSYVKRSKGRIPVKWMAIESLFDHIYTTQSDVWSFGVLLWEIVTLGGNPYPGIPPERLFNLLKTGYRMERPDNCSEEMYGLMLQCWKQEPDKRPVFADISKDLEKMMVKNRDYLDLAASTPSDSLLYDDGLSEEETPLVDCNNAPLPRTLPSTWIENKLYGMSDPNWPEESPVPLTRADGTNSVCPRYANDSVYANWMVSPSAAQLMDAFDS

SEQ ID NO: 25 - DNA, 3195 bp, Gallus gallus
  CDS (1)..(3192)
atgcgctcggtgcggggggcggccggcctcctgctgctgtgcggggcttcctttggtctgtacttccccagaaaggagtactcagagaacgtctacattgaccagccagcaggtgcgccgctcctacgcatccacgccttgagggattcacatgggaaacagcccactttcatctgtgccagaagtctcatcatttctcgagcaagatcccatgaaaatcactggtttcaaatcagagaaaaaatgggacttctctacctcagcaagagcctagatagagaagactttaacatgctgtctgtaggaaactggatgccattatcaaaggtgatgctgtatgtcttcctctcatctcaccctttccaagagaaggaatgtgactctgctactcgtaccacagtcgtcctctctttgatcaatgctactgcaccagcttgcagttcactgtcagcaaggcagctttgcttcacagaaatggatctctcctttcacatcaaggagaataaaccccctggtacatttcatcagctccagttaccctcagttcatcatctgtgtcagaatctcagcattacctacaaactgttggcagccgaaggcctgccttttcggtacaatgagaacaccactggtgtgagtgtaacacagcgcctagatcgagaggagagagagagatatgagctgatcgccaaatgcaccgtgagagaaggcttcagggaaatggaggttgaggtgcccttcctcgtcaacgtgttagatgaagatgactctcctcccttccttcccaacggcactgacactgcagatgctgtcgtggagttcaatagaaaagagggaactgtccttgcaacgctaactgtgtatgatgcagacaccacacctatttatccccttgaaagcagcagaaagaaatacacaggaacaataattactgacgatccatggataaatgaaacatttcgggtggagcacattttcgatgaaatccacttcagcccaaatggcagccaagagagaggaactcaacacgaatacaaactggtactgaataaaagtatttcagtcacagagcatcgttccttccagttggatgttttggtgaacgacacagaatttaatggcccagaaaaatcagtgatgcttcatttcaatgtctccatccttcctgtcagcattcagtttccaaacacaacttatcgattcacagtaaacagaaatgctcaacgttttgcacaaataggaaaaatctgcatcgaaaactgtatgaagttccgtggtgtgaacatcacctacaagttactgtcccccaatgtgagctgctatgctgttggcatacttcaaggccgagaagacaaatatggaagcctttatgtgaatgattcctctgtgctacttagacctgaatgcaaagaaatacaatacaccgttcaagctacagacaagcagagcaggaaacataccaaaacccttttcactgttgtattagaaggaactctttttaaaaaagaagaggactgccctgactcttgtgctatgagtaagcaccttgctgaatgtgaagagtgtggtggcctgggagtgctaacaggaagatgccagtggagacaggggagtggaaaaggaatcaccacaaactattccacatgcaccccaagtatcaagaacttgctccagatggccacctgtgatatcgtggagagcaaagatccagctatttgcccccaggactgcacaaatggaaatattattggtggtcatgggaaaggcactggaaaccttggaattaagtcaggacatgggatttgttactgcttcccaagacagaactgttattgtgagaaagatgatatcaaggagcaattttgtgatgaagtgtgcaagactgtgatagcaggggcggtgctgttgtccttcatcatctctgtgctgctttcttcctatttcatccatcgctaccacaagaattctccaaagccacctattgcctctgcagaaatgacattccggcgcccagcccagtcatatcccatcagttattcttctactaatgtccgtcgaccttctttagactccatggagaaccaggtgtctgtagacacctttaagataccagaagatccaaagtgggaattccctcggaagaacctggttctaggcaagactcttggagaaggagaatttgggaaggttgtcaaggcaacagctttcagactcaagggaagagctggctatactactgtggctgtgaaaatgctaaaagaaaatgcttcccagagcgagctgcgagatctgctgtcagagttcaaccttttgaaacaggtgaaccatcctcacgtcatcaaactttacggagcctgcagtcaagatggcccgctctatttaattgtggaatatgctaaatatggctccttgcgcagttttctcagggaaagccgaaaagtgggaccaagctacgtgggtagtgatggcaacagaaattccagttacttggataaccccgatgagagagccttaacaatgggagatctgatatcgtttgcatggcagatatcacgagggatgcagtacctggcagaaatgaagctcgtccatcgtgacttagcagccaggaatgtgctggtggcagaagggcgcaaaatgaagatttctgattttggcctatcccgtgatgtgtatgaagaagattcctatgtcaagaggagcaagggtcggatacctgttaaatggatggccatagaatccctgtttgatcatatctatacaacacaaagtgatgtgtggtcgtttggagttctgctctgggagattgtaactctgggaggcaatccctacccaggtattgctcctgaaagactctttaacctcctaaaaacaggctacagaatggagagaccagaaaactgtagtgaagaaatgtacaacctgatgttgcgctgctggaagcaagaaccagataaaaggccaacatttgctgagatcagcaaggaactggagaaaatgatggtgaagagtagggactacttagatcttgcggcttccaccccttctgattccttactatatgatgatgggctctcagaagaggagacacctctcgtggactgtaataacgctcccctccctcgaacccttccttccacatggattgaaaacaaactctatggtagaatttcacatgcatttactagattctag

SEQ ID NO: 26 - protein, 1064 aa, Gallus gallus
MRSVRGAAGLLLLCGASFGLYFPRKEYSENVYIDQPAGAPLLRIHALRDSHGKQPTFICARSLIISRARSHENHWFQIREKMGLLYLSKSLDREDFNMLSVGNWMPLSKVMLYVFLSSHPFQEKECDSATRTTVVLSLINATAPACSSLSARQLCFTEMDLSFHIKENKPPGTFHQLQLPSVHHLCQNLSITYKLLAAEGLPFRYNENTTGVSVTQRLDREERERYELIAKCTVREGFREMEVEVPFLVNVLDEDDSPPFLPNGTDTADAVVEFNRKEGTVLATLTVYDADTTPIYPLESSRKKYTGTIITDDPWINETFRVEHIFDEIHFSPNGSQERGTQHEYKLVLNKSISVTEHRSFQLDVLVNDTEFNGPEKSVMLHFNVSILPVSIQFPNTTYRFTVNRNAQRFAQIGKICIENCMKFRGVNITYKLLSPNVSCYAVGILQGREDKYGSLYVNDSSVLLRPECKEIQYTVQATDKQSRKHTKTLFTVVLEGTLFKKEEDCPDSCAMSKHLAECEECGGLGVLTGRCQWRQGSGKGITTNYSTCTPSIKNLLQMATCDIVESKDPAICPQDCTNGNIIGGHGKGTGNLGIKSGHGICYCFPRQNCYCEKDDIKEQFCDEVCKTVIAGAVLLSFIISVLLSSYFIHRYHKNSPKPPIASAEMTFRRPAQSYPISYSSTNVRRPSLDSMENQVSVDTFKIPEDPKWEFPRKNLVLGKTLGEGEFGKVVKATAFRLKGRAGYTTVAVKMLKENASQSELRDLLSEFNLLKQVNHPHVIKLYGACSQDGPLYLIVEYAKYGSLRSFLRESRKVGPSYVGSDGNRNSSYLDNPDERALTMGDLISFAWQISRGMQYLAEMKLVHRDLAARNVLVAEGRKMKISDFGLSRDVYEEDSYVKRSKGRIPVKWMAIESLFDHIYTTQSDVWSFGVLLWEIVTLGGNPYPGIAPERLFNLLKTGYRMERPENCSEEMYNLMLRCWKQEPDKRPTFAEISKELEKMMVKSRDYLDLAASTPSDSLLYDDGLSEEETPLVDCNNAPLPRTLPSTWIENKLYGRISHAFTRF

SEQ ID NO: 27 - DNA, 1998 bp, Ornithorhynchus anatinus
  CDS (1)..(1995)
atgacggtcatgaagtttgtgaaatttgtgaaatgtgcagaacttacatattgctccagtgtcaccagttgtcctcttcacggcctgcattgcccaagagcccatccgttccccctcagagcttcagaaactggtgagcggcagaggagcggagcgtgcggcgtgggcctgcaactactattattactaatgtttccattttgtggaagctctgccctgggcctctatttcccccgggatacgtactcagagaagctgtacgttgaccagccggccggcacgcccctgctctacgtccaggccttgcgggattctcagcaggaggttccacgcttccagctggggcagcacctgtacggcagttaccgttcccagctccacgagaatctctggttccagatccatgaagacacaggacttctctacgtcaacaggagtctgggctggaaggacttgaagagcctacgcgcccttaacggaggatatcctttactaaacatccacctccatgttttcctttcccccaaacctttctgggagaacgagtgcctctggcctagctgcgccaaagtgtctctctccatcatcaacaccaccgccccaacctgcagctctctgaaggcgagggagttctgcttcctggatacgggcctctctttccgcatcaaggagaacaagcctcctggcacattccaccagttccgtctcctccctgtgcagcagctctgtcccaacatcagcatctcctaccagctgatagcagtcttaatccccgttttacagttgaaggaagatccaaaatgggagtttcctcggaagaacctggttcttggaaaaactcttggggaaggcgagtttgggaaagttgtcaaagctacagccttccgactcaagggaaaagctggctacaccaccgtggctgtgaaaatgctgaaagaaaatgcatcccagagtgaactccgtgatctgctgtcagagttcaacctcctgaaacaggtcaaccatccacacgtcattaaactctacggagcttgcagtcaggatgggcctctctacctgattgtggagtatgccaaatacggctcccttcggagctttctcagggaaagccggaaggtggggcccagctacgtgggcagtgatggcaatcggaattcgagctacttggacaaccctgatgagagggcactgacaatgggagacttgatctcattcgcctggcagatctctcgaggaatgcaatatcttgcagaaatgaagctggttcatcgtgatctcgctgctagaaacgtgctggtggcagaaggacgtaaaatgaagatttcagattttggcttgtctagggatgtgtacgaagaggactcctatgtcaagaggagcaagggtcgaattcctgtcaagtggatggcgattgagtctctttttgatcatatctacactacccaaagtgatgtgtggtcatttggtgttctactctgggagatcgtgaccctcggcggaaacccttatcctgggattgctccagaacgactctttaacctcctgaaaactggttacagaatggaaaggcctgaaaactgcagtgaggaaatgtacaatttgatgttgcggtgctggaagcaagaaccagataagagaccaacctttgcagagattagcaaagaactagagaaaatgatggtgaaaagtagggactacttggatcttgctgcttccaccccatccgactccttactctatgatgacggcctctccgaagaggagacacccctcgtggactgtaataatgctcccctccctcgaacccttccttccacatggattgaaaacaaactctatggcatgtcatacccgaactggcctgaggagagtcctgtaccactcacaagattcgatggcactaactctgtgttttcaagatatgcaaatgatagtgtatatgctaactggatggtttcaccctcagcggcaaaatttatggacaagtttgatagttaa

SEQ ID NO: 28 - protein, 665 aa, Ornithorhynchus anatinus
MTVMKFVKFVKCAELTYCSSVTSCPLHGLHCPRAHPFPLRASETGERQRSGACGVGLQLLLLLMFPFCGSSALGLYFPRDTYSEKLYVDQPAGTPLLYVQALRDSQQEVPRFQLGQHLYGSYRSQLHENLWFQIHEDTGLLYVNRSLGWKDLKSLRALNGGYPLLNIHLHVFLSPKPFWENECLWPSCAKVSLSIINTTAPTCSSLKAREFCFLDTGLSFRIKENKPPGTFHQFRLLPVQQLCPNISISYQLIAVLIPVLQLKEDPKWEFPRKNLVLGKTLGEGEFGKVVKATAFRLKGKAGYTTVAVKMLKENASQSELRDLLSEFNLLKQVNHPHVIKLYGACSQDGPLYLIVEYAKYGSLRSFLRESRKVGPSYVGSDGNRNSSYLDNPDERALTMGDLISFAWQISRGMQYLAEMKLVHRDLAARNVLVAEGRKMKISDFGLSRDVYEEDSYVKRSKGRIPVKWMAIESLFDHIYTTQSDVWSFGVLLWEIVTLGGNPYPGIAPERLFNLLKTGYRMERPENCSEEMYNLMLRCWKQEPDKRPTFAEISKELEKMMVKSRDYLDLAASTPSDSLLYDDGLSEEETPLVDCNNAPLPRTLPSTWIENKLYGMSYPNWPEESPVPLTRFDGTNSVFSRYANDSVYANWMVSPSAAKFMDKFDS

SEQ ID NO: 29 - DNA, 3180 bp, Xenopus laevis
  CDS (1)..(3177)
atggcacctgcagagtgggcactctgttttctctgcctctttcaagtgacctctggactttactttctaagaaaggactattatgaaaatgtatatgtggatcaaccggcgggcacactacttctacaggtccatgtgatgaaggactcaccatcggagaaacctcactttagactctgcccaacaattaacttcagtagaaactctttttaccattggtttcggatagatgagcatacagggaacctctacctcaaccggagtcttgacaggagcgactatgaagtattctcccaaggaagtcaatcagctattcagaagataaccctgaaggtgtcaacaaagccacatccatttttggataaggactgtaataatgcacccttaacttgggttcatcttaactttataaaccttacatcttccatctgtttctcacttaaaccaaaggatctgtgcttcaatgacaaagatctctcattccacataaaagaaaacaaacctcctggaaaattttacaaatttcacccccggcctattgttcagtcctgtccaaatgtcagtgtgagttacatgctaatttcagaccacagctacccattcaggttcaatcctaatacctctgaagtgagccttacgcactctgtggaccgcgaggagagggagaattatgatcttgtggcaaaatgtttgctgagggatgcatcctcagaggtggaggtagaacaatcttttcagatcaaagtggatgatgaagatgacacagccccattcctcccgaatggaacagacactgctcatgtggtggtggacttcaaaagaaagaatgggaccgtccttggcatgctgactgtatgggatgctgactccacacctgtgtatcctgcagatgtcagtcgtaaaaaatacacaaagaccattgtcagcaaagaccggttcattctggaaaacttccgggtggagcacagctttaaggaggtgaaattccagccaaaaggcaacttaatccgtggaacacttcatgaatacaggttagttctgaacaagagtattcctattatcagaaatggatctctgcttgttagtgttctggtgaatgacactgaatttcatggaccaaatggagatgttatgttgtacttcaatgtgacaatcttatcgatggaaattagattcccaaacatctcataccagtatactgtcaaccgaaatgcagaccgatatgctcagattggaaaaatctgcatagaaaattgcaagcttgttgaaggcgtcaacattaactacagactgcaaacagaaaataacactcatgcctttgccattattcaaggaagagagagcacatttgctactcttattgtcaatgattctgatgctctggccacccatgagagtaaggaaatagtctgcactgtatttgccacaaacaagcatctgaaggaaccagtcaggacacagatcattatagtgttagaaggaacacctatgaaagatgaagataattgtccccattcatgttctgagagcaggcaaaagtctgaatgtgaggagtgtgggggactgggctcccttactggaaggtgccagtggagacagggtcatggagaaggaatcacaacaaactattctacctgcacgcctagtcttctgacatgccctgatggaatctgtgatgctgtggagatgaatgattcagcaatatgtccacaggactgcactgagataactatcattggtgggcatgaaaaaggacgtctaaaagggataaaagctggatatggcacctgtttttgccttctgcctgataaatgcttctgtgaaaagaatattgttgaaggacaactgtgtgatgacacctgcaagactgtgattgcatgtggggtctttctgtccttcataatctctgttctactctcctcatacttcattcatcgataccataaaaactctccaaagcccccaattgcttctgctgaaatgaccttccgccgccctgcccagtcttatccagtgagttactcttcaaacaatgccagacgcccatctctagactccatggagaatcagatgtcagtggatacatttaaaataccggaggatccaaagtgggagttcccacgtaaaaacctggtcttgggtaagacgcttggtgaaggagagtttggaaaagttgtaaaggcaactgcatttagactgaagggtaaagctggctacactacagtggcagtaaaaatgctgaaagagagtgcatcccagagtgagctgcgagatcttctgtcagagttcaatctcttgaaacaggtcaatcatcctcatgtcatcaaactttatggggcttgtacacaggatggtcctttgcatctcattgtagaatattcaaagtatggatccctacgcagttttctaagggaaagtcgcaaagtagggcctagcactttgggaggagattcaaatcgtaactccagctacctggataacccagatgaaagggcacttacaatgggggatctaatttcatttgcttggcagatatccaggggaatgcagtatctggcagaaatgaagcttgtccatcgagacttggcggcccgaaatgtgttggtcgcagaaggacgcaaaatgaaaatatcagattttggtctttccagagatgtttatgaagaagattcctatgtaaagagaagcaagggtagaattcctgtaaaatggatggctatagaatccctgtttgatcacatctatacaacacaaagtgacgtgtggtcgtttggagtgctcctgtgggagattgttaccttgggaggaaatccatatccgggtattgctccggagaggctctttaatctcctaaaaactggatatagaatggaaaaaccagagaactgcagtgatgaaatgtacaatcttatgcttaagtgctggaaacaagagccagaaaagagacaaacatttggcgaaatcagtaaggaactggagaagatgatggtgaaaagcagggattatttggacttggctgcatccactcctgctgactccttactgtatgacgatggtctttctgaagaggagacccctctggtggactgcaataatgccccacttcctcgaactctcccttctacctggatagaaaacaaactctatggtagaatctcccatgcgtttactagattctag

SEQ ID NO: 30 - protein, 1059 aa, Xenopus laevis
MAPAEWALCFLCLFQVTSGLYFLRKDYYENVYVDQPAGTLLLQVHVMKDSPSEKPHFRLCPTINFSRNSFYHWFRIDEHTGNLYLNRSLDRSDYEVFSQGSQSAIQKITLKVSTKPHPFLDKDCNNAPLTWVHLNFINLTSSICFSLKPKDLCFNDKDLSFHIKENKPPGKFYKFHPRPIVQSCPNVSVSYMLISDHSYPFRFNPNTSEVSLTHSVDREERENYDLVAKCLLRDASSEVEVEQSFQIKVDDEDDTAPFLPNGTDTAHVVVDFKRKNGTVLGMLTVWDADSTPVYPADVSRKKYTKTIVSKDRFILENFRVEHSFKEVKFQPKGNLIRGTLHEYRLVLNKSIPIIRNGSLLVSVLVNDTEFHGPNGDVMLYFNVTILSMEIRFPNISYQYTVNRNADRYAQIGKICIENCKLVEGVNINYRLQTENNTHAFAIIQGRESTFATLIVNDSDALATHESKEIVCTVFATNKHLKEPVRTQIIIVLEGTPMKDEDNCPHSCSESRQKSECEECGGLGSLTGRCQWRQGHGEGITTNYSTCTPSLLTCPDGICDAVEMNDSAICPQDCTEITIIGGHEKGRLKGIKAGYGTCFCLLPDKCFCEKNIVEGQLCDDTCKTVIACGVFLSFIISVLLSSYFIHRYHKNSPKPPIASAEMTFRRPAQSYPVSYSSNNARRPSLDSMENQMSVDTFKIPEDPKWEFPRKNLVLGKTLGEGEFGKVVKATAFRLKGKAGYTTVAVKMLKESASQSELRDLLSEFNLLKQVNHPHVIKLYGACTQDGPLHLIVEYSKYGSLRSFLRESRKVGPSTLGGDSNRNSSYLDNPDERALTMGDLISFAWQISRGMQYLAEMKLVHRDLAARNVLVAEGRKMKISDFGLSRDVYEEDSYVKRSKGRIPVKWMAIESLFDHIYTTQSDVWSFGVLLWEIVTLGGNPYPGIAPERLFNLLKTGYRMEKPENCSDEMYNLMLKCWKQEPEKRQTFGEISKELEKMMVKSRDYLDLAASTPADSLLYDDGLSEEETPLVDCNNAPLPRTLPSTWIENKLYGRISHAFTRF

SEQ ID NO: 31 - DNA, 38 bp, Artificial
  Synthetic DNA
gaggtcgacgccaccatggcgaaagcgaggtccggcgc

SEQ ID NO: 32 - DNA, 23 bp, Artificial
  Synthetic DNA
acggagacagtcctgggggcaaa

SEQ ID NO: 33 - DNA, 23 bp, Artificial
  Synthetic DNA
tctcctagcaccaggacctgtcc

SEQ ID NO: 34 - DNA, 35 bp, Artificial
  Synthetic DNA
gaggcggccgcgctatcaaatgtgtccattaattt